SEQUENCE LISTING (135 sequences)

SEQ ID NO: 1 - DNA, 60 bp, Artificial Sequence
  probe sequence (5′->3′)
gcatggctgctgagatcgttccacagtatgaatctctactccgcgtacagccggcaccgg

SEQ ID NO: 2 - DNA, 39 bp, Artificial Sequence
  probe sequence (5′->3′)
cagcagggcaggcagcgccaacatcgatgcgaacgtgcg

SEQ ID NO: 3 - DNA, 61 bp, Artificial Sequence
  probe sequence (5′->3′)
gcatggctgctgagatcgttccacagtatgaatctctcccggacctgcgccggcccctgcg

SEQ ID NO: 4 - DNA, 45 bp, Artificial Sequence
  probe sequence (5′->3′)
cctccagcaggccgctcagtcgccgcggatcgatgcgaacgtgcg

SEQ ID NO: 5 - DNA, 62 bp, Artificial Sequence
  Probe sequence (5′->3′)
gcatggctgctgagatcgttccacagtatgaatctctgggacagagcgcaggatcctctgcg

SEQ ID NO: 6 - DNA, 39 bp, Artificial Sequence
  Probe sequence (5′->3′)
cttgcggactgggagcgggcggatcgatgcgaacgtgcg

SEQ ID NO: 7 - DNA, 60 bp, Artificial Sequence
  Probe sequence (5′->3′)
gcatggctgctgagatcgttccacagtatgaatctctcccagccttctgggcaggcgcat

SEQ ID NO: 8 - DNA, 36 bp, Artificial Sequence
  Probe sequence (5′->3′)
ggccgggcggcgcccagccctcgatgcgaacgtgcg

SEQ ID NO: 9 - DNA, 60 bp, Artificial Sequence
  Probe sequence (5′->3′)
gcatggctgctgagatcgttccacagtatgaatctctcgcgtttcccggggaaaagcgga

SEQ ID NO: 10 - DNA, 47 bp, Artificial Sequence
  Probe sequence (5′->3′)
tcccggagaagcagcctaatctctcagcccttcgatgcgaacgtgcg

SEQ ID NO: 11 - DNA, 60 bp, Artificial Sequence
  Probe sequence (5′->3′)
gcatggctgctgagatcgttccacagtatgaatctctccgtgggtgagtgggagggtccg

SEQ ID NO: 12 - DNA, 41 bp, Artificial Sequence
  Probe sequence (5′->3′)
ggactgcagaccggtggcgatggcctcgatgcgaacgtgcg

SEQ ID NO: 13 - DNA, 60 bp, Artificial Sequence
  Probe sequence (5′->3′)
gcatggctgctgagatcgttccacagtatgaatctctggccgtgcatctgcgcaacgctg

SEQ ID NO: 14 - DNA, 39 bp, Artificial Sequence
  Probe sequence (5′->3′)
gcttgggagccggccggtggtggtcgatgcgaacgtgcg

SEQ ID NO: 15 - DNA, 60 bp, Artificial Sequence
  Probe sequence (5′->3′)
gcatggctgctgagatcgttccacagtatgaatctcttccagcttccgcgtacctgcgcg

SEQ ID NO: 16 - DNA, 38 bp, Artificial Sequence
  Probe sequence (5′->3′)
cacaccgggtcccccgcggccttcgatgcgaacgtgcg

SEQ ID NO: 17 - DNA, 59 bp, Artificial Sequence
  Probe sequence (5′->3′)
gcatggctgctgagatcgttccacagtatgaatctctcgccccggagcgggcgcgtcct

SEQ ID NO: 18 - DNA, 41 bp, Artificial Sequence
  Probe sequence (5′->3′)
tctggaccacccaggcttggcgaggtcgatgcgaacgtgcg

SEQ ID NO: 19 - DNA, 60 bp, Artificial Sequence
  Probe sequence (5′->3′)
gcatggctgctgagatcgttccacagtatgaatctctccaatgcccttctccgcgctcct

SEQ ID NO: 20 - DNA, 39 bp, Artificial Sequence
  Probe sequence (5′->3′)
agcgcggttactgggcgctgccctcgatgcgaacgtgcg

SEQ ID NO: 21 - DNA, 59 bp, Artificial Sequence
  Probe sequence (5′->3′)
gcatggctgctgagatcgttccacagtatgaatctctgcccccgtcgtgcccgtgctcc

SEQ ID NO: 22 - DNA, 41 bp, Artificial Sequence
  Probe sequence (5′->3′)
ggagcgctagtctccgccacgaacgtcgatgcgaacgtgcg

SEQ ID NO: 23 - DNA, 64 bp, Artificial Sequence
  Probe sequence (5′->3′)
gcatggctgctgagatcgttccacagtatgaatctctccggtgtcgtcccccatcgttacgcag

SEQ ID NO: 24 - DNA, 40 bp, Artificial Sequence
  Probe sequence (5′->3′)
cgcgttcgtgccagggcaggtctgtcgatgcgaacgtgcg

SEQ ID NO: 25 - DNA, 60 bp, Artificial Sequence
  Probe sequence (5′->3′)
gcatggctgctgagatcgttccacagtatgaatctctgctggatcccgggcctgcggagt

SEQ ID NO: 26 - DNA, 40 bp, Artificial Sequence
  Probe sequence (5′->3′)
ctgcccggaaaggccacaggaggctcgatgcgaacgtgcg

SEQ ID NO: 27 - DNA, 59 bp, Artificial Sequence
  Probe sequence (5′->3′)
gcatggctgctgagatcgttccacagtatgaatctctgcagccgctccagcaggcgccg

SEQ ID NO: 28 - DNA, 42 bp, Artificial Sequence
  Probe sequence (5′->3′)
gtagtagcgcagcccctcgcggttggtcgatgcgaacgtgcg

SEQ ID NO: 29 - DNA, 59 bp, Artificial Sequence
  Probe sequence (5′->3′)
gcatggctgctgagatcgttccacagtatgaatctctgtgagcgcgttcctcggcggcg

SEQ ID NO: 30 - DNA, 39 bp, Artificial Sequence
  Probe sequence (5′->3′)
cggcgcggcgctctgggtcctcctcgatgcgaacgtgcg

SEQ ID NO: 31 - DNA, 62 bp, Artificial Sequence
  Probe sequence (5′->3′)
gcatggctgctgagatcgttccacagtatgaatctctgtgcgagcactaccggtggaggagc

SEQ ID NO: 32 - DNA, 39 bp, Artificial Sequence
  Probe sequence (5′->3′)
tgcgcgtctattgcgccctgctgtcgatgcgaacgtgcg

SEQ ID NO: 33 - DNA, 59 bp, Artificial Sequence
  Probe sequence (5′->3′)
gcatggctgctgagatcgttccacagtatgaatctctacctgcgcgctccgcctggcgc

SEQ ID NO: 34 - DNA, 41 bp, Artificial Sequence
  Probe sequence (5′->3′)
gcacaccgggctagggcgtctctggtcgatgcgaacgtgcg

SEQ ID NO: 35 - DNA, 59 bp, Artificial Sequence
  Probe sequence (5′->3′)
gcatggctgctgagatcgttccacagtatgaatctctcgaccagggccaggcccagcgc

SEQ ID NO: 36 - DNA, 40 bp, Artificial Sequence
  Probe sequence (5′->3′)
cagcgggaggttcgaacgcgccattcgatgcgaacgtgcg

SEQ ID NO: 37 - DNA, 60 bp, Artificial Sequence
  Probe sequence (5′->3′)
gcatggctgctgagatcgttccacagtatgaatctcttcctgcgactggagcgcgagcgg

SEQ ID NO: 38 - DNA, 39 bp, Artificial Sequence
  Probe sequence (5′->3′)
agctgccccgcggcttcgccacatcgatgcgaacgtgcg

SEQ ID NO: 39 - DNA, 63 bp, Artificial Sequence
  Probe sequence (5′->3′)
gcatggctgctgagatcgttccacagtatgaatctctgctctgcaccttcctcccccagcgct

SEQ ID NO: 40 - DNA, 41 bp, Artificial Sequence
  Probe sequence (5′->3′)
ttcttcccgcgcccgtcgaatcctctcgatgcgaacgtgcg

SEQ ID NO: 41 - DNA, 60 bp, Artificial Sequence
  Probe sequence (5′->3′)
gcatggctgctgagatcgttccacagtatgaatctctacccggccccgccggccatgagg

SEQ ID NO: 42 - DNA, 40 bp, Artificial Sequence
  Probe sequence (5′->3′)
cgcgcgccttccctggtccttttctcgatgcgaacgtgcg

SEQ ID NO: 43 - DNA, 60 bp, Artificial Sequence
  Probe sequence (5′->3′)
gcatggctgctgagatcgttccacagtatgaatctctcccaacggcccccgggagctctc

SEQ ID NO: 44 - DNA, 42 bp, Artificial Sequence
  Probe sequence (5′-3′)
gcgagattccggcatctctcaccccgtcgatgcgaacgtgcg

SEQ ID NO: 45 - DNA, 60 bp, Artificial Sequence
  Probe sequence (5′->3′)
gcatggctgctgagatcgttccacagtatgaatctctatgcagtcccgggtcgggagccc

SEQ ID NO: 46 - DNA, 39 bp, Artificial Sequence
  Probe sequence (5′->3′)
caggccgggcgcgcgggtgtagatcgatgcgaacgtgcg

SEQ ID NO: 47 - DNA, 59 bp, Artificial Sequence
  Probe sequence (5′->3′)
gcatggctgctgagatcgttccacagtatgaatctcttgcgccgggcggctgcgcgtcc

SEQ ID NO: 48 - DNA, 40 bp, Artificial Sequence
  Probe sequence (5′->3′)
cggatgcgtccgcggcagaagatgtcgatgcgaacgtgcg

SEQ ID NO: 49 - DNA, 60 bp, Artificial Sequence
  Probe sequence (5′->3′)
gcatggctgctgagatcgttccacagtatgaatctctcgagggcaaagcgctctcggcgg

SEQ ID NO: 50 - DNA, 40 bp, Artificial Sequence
  Probe sequence (5′>3′)
tgcggtgtgagccacctcccggcgtcgatgcgaacgtgcg

SEQ ID NO: 51 - DNA, 60 bp, Artificial Sequence
  Probe sequence (5′->3′)
gcatggctgctgagatcgttccacagtatgaatctctatactgccgcgggtcggcgtccg

SEQ ID NO: 52 - DNA, 47 bp, Artificial Sequence
  Probe sequence (5′->3′)
catcgacgcgctttagaaacggctctaggtttcgatgcgaacgtgcg

SEQ ID NO: 53 - DNA, 60 bp, Artificial Sequence
  Probe sequence (5′->3′)
gcatggctgctgagatcgttccacagtatgaatctctcggcgcggatcgacggtgaagcg

SEQ ID NO: 54 - DNA, 40 bp, Artificial Sequence
  Probe sequence (5′->3′)
cggcgccgacagggcggccgagattcgatgcgaacgtgcg

SEQ ID NO: 55 - DNA, 60 bp, Artificial Sequence
  Probe sequence (5′->3′)
gcatggctgctgagatcgttccacagtatgaatctctcgggcccggaggcccagccccgc

SEQ ID NO: 56 - DNA, 39 bp, Artificial Sequence
  Probe sequence (5′->3′)
gccaagggccacccctcggcgcgtcgatgcgaacgtgcg

SEQ ID NO: 57 - DNA, 60 bp, Artificial Sequence
  Probe sequence (5′->3′)
gcatggctgctgagatcgttccacagtatgaatctctaacaccggcgctggcagtggcgg

SEQ ID NO: 58 - DNA, 40 bp, Artificial Sequence
  Probe sequence (5′->3′)
cgcgggaggaaagccggcttcctgtcgatgcgaacgtgcg

SEQ ID NO: 59 - DNA, 60 bp, Artificial Sequence
  Probe sequence (5′->3′)
gcatggctgctgagatcgttccacagtatgaatctctctgcagggcgtggagaccccgcc

SEQ ID NO: 60 - DNA, 38 bp, Artificial Sequence
  Probe sequence (5′->3′)
cggcgcagaacgcgctggccagtcgatgcgaacgtgcg

SEQ ID NO: 61 - DNA, 60 bp, Artificial Sequence
  Probe sequence (5′->3′)
gcatggctgctgagatcgttccacagtatgaatctctgcgcccgcaggcccggccgccgc

SEQ ID NO: 62 - DNA, 42 bp, Artificial Sequence
  Probe sequence (5′->3′)
gctgccgtctcgcaccccatccgcgctcgatgcgaacgtgcg

SEQ ID NO: 63 - DNA, 60 bp, Artificial Sequence
  Probe sequence (5->3′)
gcatggctgctgagatcgttccacagtatgaatctctgcggctcacccgagacccggcgc

SEQ ID NO: 64 - DNA, 39 bp, Artificial Sequence
  Probe sequence (5′->3′)
gcccttcctgccggaccctcggctcgatgcgaacgtgcg

SEQ ID NO: 65 - DNA, 59 bp, Artificial Sequence
  Probe sequence (5′->3′)
gcatggctgctgagatcgttccacagtatgaatctctggctggcggcgcggtgccaagg

SEQ ID NO: 66 - DNA, 39 bp, Artificial Sequence
  Probe sequence (5′->3′)
cgccccggctccagggctctgcgtcgatgcgaacgtgcg

SEQ ID NO: 67 - DNA, 59 bp, Artificial Sequence
  Probe sequence (5′->3′)
gcatggctgctgagatcgttccacagtatgaatctctattccccggcttcgccggacgc

SEQ ID NO: 68 - DNA, 36 bp, Artificial Sequence
  Probe sequence (5′->3′)
cacaggcagagtgccgcgggtcgatgcgaacgtgcg

SEQ ID NO: 69 - DNA, 59 bp, Artificial Sequence
  Probe sequence (5′->3′)
gcatggctgctgagatcgttccacagtatgaatctctgcagcagcgctccacaccgcgg

SEQ ID NO: 70 - DNA, 41 bp, Artificial Sequence
  Probe sequence (5′->3′)
ctgcagcggctccgggttaatcagctcgatgcgaacgtgcg

SEQ ID NO: 71 - DNA, 60 bp, Artificial Sequence
  Probe sequence (5′->3′)
gcatggctgctgagatcgttccacacatagagttcttgcagcctggccgctcgctgaggc

SEQ ID NO: 72 - DNA, 39 bp, Artificial Sequence
  Probe sequence (5′->3′)
gcggagagcgcggaacgagcgcgtcgatgcgaacgtgcg

SEQ ID NO: 73 - DNA, 58 bp, Artificial Sequence
  Probe sequence (5′->3′)
gcatggctgctgagatcgttccacacatagagttcttctggacgcgccgagagccgcg

SEQ ID NO: 74 - DNA, 38 bp, Artificial Sequence
  Probe sequence (5′->3′)
gcccgcccaccgggtcacatggtcgatgcgaacgtgcg

SEQ ID NO: 75 - DNA, 59 bp, Artificial Sequence
  Probe sequence (5′->3′)
gcatggctgctgagatcgttccacacatagagttcttagacctgcgcccgcgaagccac

SEQ ID NO: 76 - DNA, 39 bp, Artificial Sequence
  Probe sequence (5′->3′)
tccggccagcggcgccgcccgcttcgatgcgaacgtgcg

SEQ ID NO: 77 - DNA, 60 bp, Artificial Sequence
  Probe sequence (5′->3′)
gcatggctgctgagatcgttccacacatagagttcttagctcccgcctagccccggacgc

SEQ ID NO: 78 - DNA, 38 bp, Artificial Sequence
  Probe sequence (5′->3′)
gcccacctgggcggctcctccctcgatgcgaacgtgcg

SEQ ID NO: 79 - DNA, 58 bp, Artificial Sequence
  Probe sequence (5′->3′)
gcatggctgctgagatcgttccacacatagagttcttggccgaggccgccttcgccgc

SEQ ID NO: 80 - DNA, 37 bp, Artificial Sequence
  Probe sequence (5′->3′)
gcgcgttcccggggccccgcctcgatgcgaacgtgcg

SEQ ID NO: 81 - DNA, 60 bp, Artificial Sequence
  Probe sequence (5′->3′)
gcatggctgctgagatcgttccacacatagagttcttctgacgccccagccagagaccgc

SEQ ID NO: 82 - DNA, 39 bp, Artificial Sequence
  Probe sequence (5′->3′)
gcccggtcagggagcagggtccatcgatgcgaacgtgcg

SEQ ID NO: 83 - DNA, 60 bp, Artificial Sequence
  Probe sequence (5′->3′)
gcatggctgctgagatcgttccacacatagagttcttgcacaccggcgtgcgcgccttcc

SEQ ID NO: 84 - DNA, 44 bp, Artificial Sequence
  Probe sequence (5′->3′)
agtgcgccgtctgcgccaagcgcttcactcgatgcgaacgtgcg

SEQ ID NO: 85 - DNA, 59 bp, Artificial Sequence
  Probe sequence (5′->3′)
gcatggctgctgagatcgttccacacatagagttcttcgcgcgctcgggccggtgagtg

SEQ ID NO: 86 - DNA, 42 bp, Artificial Sequence
  Probe sequence (5′->3′)
cgcatgtgcacgttgagcgagctctttcgatgcgaacgtgcg

SEQ ID NO: 87 - DNA, 60 bp, Artificial Sequence
  Probe sequence (5′->3′)
gcatggctgctgagatcgttccacacatagagttctttgacccgacggccaggcggtggc

SEQ ID NO: 88 - DNA, 43 bp, Artificial Sequence
  Probe sequence (5′->3′)
gcaggggccagataaggctcttccggctcgatgcgaacgtgcg

SEQ ID NO: 89 - DNA, 60 bp, Artificial Sequence
  Probe sequence (5′->3′)
gcatggctgctgagatcgttccacacatagagttcttgacggcagcgggctccttcccgc

SEQ ID NO: 90 - DNA, 40 bp, Artificial Sequence
  Probe sequence (5′->3′)
gccccaggtcatgtgcagcccctgtcgatgcgaacgtgcg

SEQ ID NO: 91 - DNA, 60 bp, Artificial Sequence
  Probe sequence (5′->3′)
gcatggctgctgagatcgttccacacatagagttcttagcggaaccccgccccggccagc

SEQ ID NO: 92 - DNA, 41 bp, Artificial Sequence
  Probe sequence (5′->3′)
gcggagtagggaggagtggagggcgtcgatgcgaacgtgcg

SEQ ID NO: 93 - DNA, 58 bp, Artificial Sequence
  Probe sequence (5′->3′)
gcatggctgctgagatcgttccacacatagagttcttcgcctgcccctcgccagcgcg

SEQ ID NO: 94 - DNA, 45 bp, Artificial Sequence
  Probe sequence (5′->3′)
cagatcgccagtccctcagtttgcccggctcgatgcgaacgtgcg

SEQ ID NO: 95 - DNA, 60 bp, Artificial Sequence
  Probe sequence (5′->3′)
gcatggctgctgagatcgttccacacatagagttcttcctcgtcccccactgtggccgcg

SEQ ID NO: 96 - DNA, 41 bp, Artificial Sequence
  Probe sequence (5′->3′)
ccagctccatcccgggccagccgcgtcgatgcgaacgtgcg

SEQ ID NO: 97 - DNA, 59 bp, Artificial Sequence
  Probe sequence (5′->3′)
gcatggctgctgagatcgttccacacatagagttcttagccccacgcggcgcaagaacc

SEQ ID NO: 98 - DNA, 35 bp, Artificial Sequence
  Probe sequence (5′->3′)
ccgggcgctgagctccgggtcgatgcgaacgtgcg

SEQ ID NO: 99 - DNA, 60 bp, Artificial Sequence
  Probe sequence (5′->3′)
gcatggctgctgagatcgttccacacatagagttcttacgccgggcgcgcgaactacact

SEQ ID NO: 100 - DNA, 39 bp, Artificial Sequence
  Probe sequence (5′->3′)
tcccagaggcccgcgcgcgcaggtcgatgcgaacgtgcg

SEQ ID NO: 101 - DNA, 60 bp, Artificial Sequence
  Probe sequence (5′->3′)
gcatggctgctgagatcgttccacacatagagttcttgacacacacgctcgcgcccgcgc

SEQ ID NO: 102 - DNA, 47 bp, Artificial Sequence
  Probe sequence (5′->3′)
ctcctcgtctgcacttcaaagcgagtggcgctcgatgcgaacgtgcg

SEQ ID NO: 103 - DNA, 59 bp, Artificial Sequence
  Probe sequence (5′->3′)
gcatggctgctgagatcgttccacacatagagttcttccgaggccgccttcgccgcgcg

SEQ ID NO: 104 - DNA, 39 bp, Artificial Sequence
  Probe sequence (5′->3′)
cgttcccggggccccgcccgatctcgatgcgaacgtgcg

SEQ ID NO: 105 - DNA, 60 bp, Artificial Sequence
  Probe sequence (5′->3′)
gcatggctgctgagatcgttccacacatagagttcttcccgtgccccctcttacccgggc

SEQ ID NO: 106 - DNA, 38 bp, Artificial Sequence
  Probe sequence (5′->3′)
gcctgggccgcccccgccgtcttcgatgcgaacgtgcg

SEQ ID NO: 107 - DNA, 63 bp, Artificial Sequence
  Probe sequence (5′->3′)
gcatggctgctgagatcgttccacacatagagttcttgggttggcccgacctagacttggcgc

SEQ ID NO: 108 - DNA, 40 bp, Artificial Sequence
  Probe sequence (5′->3′)
tgtatcgcggctgggcctgtcgcatcgatgcgaacgtgcg

SEQ ID NO: 109 - DNA, 60 bp, Artificial Sequence
  Probe sequence (5′->3′)
gcatggctgctgagatcgttccacacatagagttcttcgcctcggcggagagcagccccg

SEQ ID NO: 110 - DNA, 39 bp, Artificial Sequence
  Probe sequence (5′->3′)
ggcgcggcagcagcgtcagccgttcgatgcgaacgtgcg

SEQ ID NO: 111 - DNA, 63 bp, Artificial Sequence
  Probe sequence (5′->3′)
gcatggctgctgagatcgttccacacatagagttcttccccgggctggcactcgagatatgtg

SEQ ID NO: 112 - DNA, 47 bp, Artificial Sequence
  Probe sequence (5′->3′)
agcgcgtgttacgttcaactttgctttgcagtcgatgcgaacgtgcg

SEQ ID NO: 113 - DNA, 60 bp, Artificial Sequence
  Probe sequence (5′->3′)
gcatggctgctgagatcgttccacacatagagttcttccgggacccccgagtcctggctt

SEQ ID NO: 114 - DNA, 41 bp, Artificial Sequence
  Probe sequence (5′->3′)
ggcgccctcaccggtgaggagctgctcgatgcgaacgtgcg

SEQ ID NO: 115 - DNA, 60 bp, Artificial Sequence
  Probe sequence (5′-3′)
gcatggctgctgagatcgttccacacatagagttcttccggggcctgcagctcgtggagc

SEQ ID NO: 116 - DNA, 41 bp, Artificial Sequence
  Probe sequence (5′->3′)
agcgccagcagcagtggcacccagctcgatgcgaacgtgcg

SEQ ID NO: 117 - DNA, 60 bp, Artificial Sequence
  Probe sequence (5′->3′)
gcatggctgctgagatcgttccacacatagagttctttccagtccccacagcgttcgcgc

SEQ ID NO: 118 - DNA, 42 bp, Artificial Sequence
  Probe sequence (5′->3′)
tcccagccggggtaagcggaagaaaatcgatgcgaacgtgcg

SEQ ID NO: 119 - DNA, 64 bp, Artificial Sequence
  Probe sequence (5′->3′)
gcatggctgctgagatcgttccacacatagagttcttacctcaaggggagaacagaaggccggc

SEQ ID NO: 120 - DNA, 43 bp, Artificial Sequence
  Probe sequence (5′->3′)
tccgcgtggcatctagcactgtggagctcgatgcgaacgtgcg

SEQ ID NO: 121 - DNA, 63 bp, Artificial Sequence
  Probe sequence (5′->3′)
gcatggctgctgagatcgttccacacatagagttctttcagcttcatcccatgcctgtcgcgc

SEQ ID NO: 122 - DNA, 45 bp, Artificial Sequence
  Probe sequence (5′->3′)
tgccacccaaacactcatgcaccggaagttcgatgcgaacgtgcg

SEQ ID NO: 123 - DNA, 17 bp, Artificial Sequence
  Probe for Group 1 biomarker/biomarker region; Probe sequence (5′->3′)
ccacagtatgaatctct

SEQ ID NO: 124 - DNA, 17 bp, Artificial Sequence
  Probe for Group 2 biomarker/biomarker region; Probe sequence (5′->3′)
ccacacatagagttctt

SEQ ID NO: 125 - DNA, 17 bp, Artificial Sequence
  Probe for Mix10 Group 1; Probe sequence (5′->3′)
ccacagtatgaatctct

SEQ ID NO: 126 - DNA, 17 bp, Artificial Sequence
  Probe for Mix 10 Group 2; Probe sequence (5′->3′)
ccacacatagagttctt

SEQ ID NO: 127 - DNA, 19 bp, Artificial Sequence
  Forward primer; primer sequence (5′->3′)
gcatggctgctgagatcgt

SEQ ID NO: 128 - DNA, 16 bp, Artificial Sequence
  reverse primer; primer sequence (5′->3′)
cgcacgttcgcatcga

SEQ ID NO: 129 - DNA, 13 bp, Artificial Sequence
  Probe for Group 1; Probe sequence (5′->3′)
cggctgccacccg

SEQ ID NO: 130 - DNA, 14 bp, Artificial Sequence
  Probe for Group 2; probe sequence (5′->3′)
cgcgccttccagtg

SEQ ID NO: 131 - DNA, 17 bp, Artificial Sequence
  Forward primer for Group 1; primer sequence (5′->3′)
accccacagcggagctc

SEQ ID NO: 132 - DNA, 20 bp, Artificial Sequence
  Forward primer for Group 2; Primer sequence (5′->3′)
aacacatggtcacgcacacc

SEQ ID NO: 133 - DNA, 20 bp, Artificial Sequence
  Reverse primer for Group 1; primer sequence (5′->3′)
agaaaaggaccagggaaggc

SEQ ID NO: 134 - DNA, 15 bp, Artificial Sequence
  Reverse primer for Group 2; primer sequence (5′->3′)
cgcttggcgcagacg

SEQ ID NO: 135 - DNA, 16 bp, Artificial Sequence
  qPCR reverse rprimer; primer sequence (5′->3′)
tcgatgcgaacgtgcg

(19) United States
(12) Patent Application Publication
(10) Pub. No.: US 2015/0275300 A1
(43) Pub. Date: Oct. 1, 2015

(54) BIOMARKERS FOR DOWN SYNDROME PRENATAL DIAGNOSIS

(71) Applicants: AGENCY FOR SCIENCE, TECHNOLOGY AND RESEARCH, Singapore (SG); SINGAPORE HEALTH SERVICES PTE LTD, Singapore (SG)

(72) Inventors: Chunming Ding, Singapore (SG); Shengnan Jin, Singapore (SG); Yew Kok Lee, Singapore (SG); Seow Heong Yeo, Singapore (SG)

(73) Assignee: Agency for Science, Technology and Research, Singapore (SG)

(21) Appl. No.: 14/431,729

(22) PCT Filed: Sep. 26, 2013
(86) PCT No.: PCT/SG2013/000421

§ 371 Date: Mar. 26, 2015

(30) Foreign Application Priority Data
Sep. 26, 2012 (SG) 201207172-6

(51) Int. Cl. C12Q 1/68

(52) U.S. Cl. CPC C12Q 1/6883; C12Q 2600/154; C12Q 2600/158

(57) ABSTRACT

Disclosed is an isolated biomarker/biomarker region. Also disclosed are isolated biomarker/biomarker regions for detecting trisomy 21, methods of determining the likelihood of a foetus to suffer from a specific disease using the biomarker/biomarker region, a kit and a method of determining the methylation levels of a biomarker/biomarker region.

### CROSS-REFERENCE TO RELATED APPLICATIONS

This application claims the benefit of priority of Singapore patent application No. 201207172-6, filed 26 Sep. 2012, the contents of it being hereby incorporated by reference in its entirety for all purposes.

### FIELD OF THE INVENTION

The present invention relates to biochemistry in particular biomarkers/biomarker regions. In particular, the present invention relates to biomarker/biomarker regions associated with Down syndrome and methods of using the biomarkers to determine the likelihood that a foetus will have Down syndrome.

### BACKGROUND OF THE INVENTION

Current methods for screening congenital diseases in foetus include ultrasound such as foetal nuchal translucency (NT) and other tests to detect biomarkers found in maternal serum. For example, in screens for Down syndrome, biomarkers measured include the amount of alpha fetoprotein (AFP) and human chorionic gonadotropin, which are produced by the foetus and the placenta and can be detected in the maternal serum. Together with the age of the mother and results of foetal nuchal translucency scan, the measurements of alpha fetoprotein and human chorionic gonadotropin are used to calculate the risk of the baby having Down syndrome. When the resulting numerical risk is classified as high risk, to confirm the results, an invasive test using chorionic villus sampling (CVS) or amniocentesis to obtain foetal tissue is required. As chorionic villus sampling or amniocentesis involves the insertion of a fine needle into the womb, these procedures may cause miscarriage.

Down syndrome, or Mongolism, is a congenital condition caused by a defect in the chromosomes. An individual born with Down syndrome has three copies of chromosome 21, instead of the usual two, thus causing the disease to be also known as trisomy 21.

The cause of Down syndrome is unclear and no direct genotype-phenotype associations have been established. However, certain conditions such as advanced maternal age and history of having another child or previous pregnancy with Down syndrome are found to increase risk of having a foetus with Down syndrome. As an individual with Down syndrome would develop complex clinical features and symptoms such as lifelong mental retardation, development delays and other problems such as seizures, thyroid disorders, cardiac defects, an increased risk of leukaemia, infertility, gastrointestinal defects and early aging, there is a need to provide for an accurate detection of a foetus with trisomy 21.

Since the current screening markers offer low specificity and reliable screening methods rely on the collection of amniotic fluid via amniocentesis or chorionic villus sampling sample, there is a need to provide alternative methods or biomarkers that can be used to screen diseases in foetus.

### SUMMARY OF THE INVENTION

In one aspect, there is provided an isolated biomarker/biomarker region comprising a DNA region of the human genome selected from a DNA region listed in any one of tables 3 to 6 (groups 3, 4, 1′ and 2′).

In another aspect, there is provided an isolated biomarker/biomarker region for detecting trisomy 21 or partial trisomy 21, comprising a DNA region of the human genome selected from a DNA region listed in any one of tables 3 to 6 (groups 3, 4, and 2′).

In yet another aspect, there is provided an isolated biomarker/biomarker region comprising a DNA region of the human genome selected from a DNA region listed in any one of tables 1 to 4 and 7 to 8 (groups 1 to 4 and Mix10 Group 1 and Mix10 Group 2 respectively), wherein the level of DNA-methylation of any one of the biomarker/biomarker regions in a diseased sample is different from the level of DNA-methylation in the same biomarker/biomarker region of a non-diseased control DNA.

In yet another aspect there is provided a method determining the likelihood of a foetus to suffer from a specific disease. The method comprising the steps of: a) providing an isolated total DNA sample from a pregnant woman, comprising foetal DNA and maternal DNA. Further comprising the steps of: b) removing maternal DNA background; c) measuring a signal indicative for the level of foetal DNA based on one or more biomarkers/biomarker regions, where in the case where the maternal DNA background had a level of methylation below 10%, the signal is the level of methylated foetal DNA and in the case where the maternal DNA background had a level of methylation above 90%, the signal is the level of unmethylated foetal DNA; d) determining a ratio of signals obtained under step c) by dividing the signals of one or more of Group 1 and/or Group 3 biomarkers/biomarker regions over the signals of one or more of Group 2 and/or Group 4 biomarkers/biomarker regions, wherein a ratio higher than the ratio determined in control foetal DNA obtained from a non-diseased foetus indicates that the foetus is likely to suffer from the specific disease. In one example, each of the groups is characterized by:

Group 1: maternal DNA background has a level of methylation below 10% and the signal of the biomarker/biomarker region is higher in foetal DNA obtained from a foetus suffering from the specific disease compared to the same biomarker/biomarker region in control foetal DNA obtained from a foetus not suffering from the disease.

Group 2: maternal DNA background has a level of methylation below 10% and the signal of the biomarker/biomarker region is lower in foetal DNA obtained from a foetus suffering from the specific disease compared to the same biomarker/biomarker region in control foetal DNA obtained from a foetus not suffering from the disease.

Group 3: maternal DNA background has a level of methylation above 90% and the signal of the biomarker/biomarker region is higher in foetal DNA obtained from a foetus suffering from the specific disease compared to the same biomarker/biomarker region in control foetal DNA obtained from a foetus not suffering from the disease.

Group 4: maternal DNA background has a level of methylation above 90% and the signal of the biomarker/biomarker region is lower in foetal DNA obtained from a foetus suffering from the specific disease compared to the same biomarker/biomarker region in control foetal DNA obtained from a foetus not suffering from the disease.

In yet another aspect, there is provided a method of determining the likelihood of a foetus to suffer from trisomy 21 or partial trisomy 21. The method comprising the steps of: a) providing an isolated total DNA sample from a pregnant woman, comprising foetal DNA and maternal DNA; b) removing maternal DNA background; c) measuring a signal indicative for the level of foetal DNA based on one or more biomarkers/biomarker regions listed in any one of Tables 1 to 8, where in the case where the maternal DNA background had a level of methylation below 10%; the signal is the level of methylated foetal DNA and in the case where the maternal DNA background had a level of methylation above 90%, the signal is the level of unmethylated foetal DNA; d) determining a ratio of signals obtained under step c) by dividing the signals of one or more of Group 1 and/or Group 3 biomarkers/biomarker regions over the signals of one or more of Group 2 and/or Group 4 biomarkers/biomarker regions, wherein a ratio higher than the ratio determined in control foetal DNA obtained from a non-diseased foetus indicates that the foetus is likely to suffer from trisomy 21 or partial trisomy 21. In one example, each of the groups is characterized by:

Group 1: biomarker/biomarker region listed in Table 1 (Group 1), Table 5 (Group 1′), or Table 7 (Mix10 Group 1).

Group 2: biomarker/biomarker region listed in Table 2 (Group 2) or Table 6 (Group 2′), or Table 8 (Mix10 Group 2).

Group 3: biomarker/biomarker region listed in Table 3 (Group 3).

Group 4: biomarker/biomarker region listed in Table 4 (Group 4).

In yet another aspect there is provided a kit comprising primers for amplifying the one or more biomarkers/biomarker regions selected from any one of the DNA regions of the human genome listed in any one of tables 3 to 6 (groups 3, 4, 1′ and 2′). The kit further comprises one or more reagents for measuring a signal indicative for the level of foetal DNA based on the one or more biomarkers/biomarker regions.

In yet another aspect there is provided a kit comprising primers for amplifying the one or more biomarkers/biomarker regions selected from any one of the DNA regions of the human genome listed in any one of tables 1 to 4 (groups 1 to 4). The kit further comprises one or more reagents for measuring a signal indicative for the level of foetal DNA based on the one or more biomarkers/biomarker regions.

In yet another aspect there is provided a kit comprising primers for amplifying the one or more biomarkers/biomarker regions selected from any one of the DNA regions of the human genome listed in any one of tables 7 to 8 (Mix10 Group 1 and Mix10 Group 2). The kit further comprises one or more reagents for measuring a signal indicative for the level of foetal DNA based on the one or more biomarkers/biomarker regions.

In yet another aspect there is provided a method of determining the methylation levels of a biomarker/biomarker region comprising the steps of a) treating a sample comprising both foetal and maternal DNA with a reagent that differentially modifies methylated and non-methylated DNA. The method further comprises b) calculating the percentage of unmodified cytosine residues over the total number of modified and unmodified cytosine residues in order to determine the methylation levels of a biomarker/biomarker region.

### BRIEF DESCRIPTION OF THE DRAWINGS

The invention will be better understood with reference to the detailed description when considered in conjunction with the non-limiting examples and the accompanying drawings.

FIG. 1: Schematic illustration of the steps performed in Example 2, whereby a trisomy 21 (T21) foetus detection using methylation biomarkers is performed. The steps are explained in detail in the description. The result of performing these steps is a differentiation between a trisomy 21 foetus and a normal foetus by quantifying the foetal-specific DNA, utilizing a universal qPCR primer pair and group specific probes. If the ratio between group 1 and group 2 is high, then the foetus is deemed to be trisomy 21; if the ratio is low, then the foetus is deemed to be normal. The group specific probes are defined according to tables 7 and 8. FIG. 1 illustrates one example of the application of the method of the present disclosure in determining the likelihood of a foetus having trisomy 21.

FIG. 2: Signal difference between Group 1 and Group 2 biomarkers with probe mix10. This histogram shows data resulting from a DNA analysis using Group 1 and Group 2 biomarkers on trisomy 21 (T21) and normal samples. The difference shown here is the result of different methylations between the trisomy 21 and the normal group when using probe mix10, which, contains 35 biomarkers from Group 1 and 26 biomarkers from Group 2. The details of probe sequences and their target biomarkers are listed in Mix10 Group 1 (see Table 7) and Mix10 Group 2 (see Table 8). Thus, FIG. 2 demonstrates an exemplary data that may be obtained from a method of the present disclosure, showing samples obtained from trisomy 21 are markedly different from sample obtained from normal individual.

FIG. 3: Signal difference between normal and trisomy 21 (T21) foetal DNA with probe mix10. This histogram here visualizes the difference in the signal intensity between trisomy 21 and normal tissues. ΔΔCt (Group2−Group1) values from probe mix10, whose methylation difference between normal and trisomy 21 tissues is the biggest among all combinations of probe mixtures tested, is shown. Thus, FIG. 3 shows clear differences observed in signals obtained from samples analysed using the biomarker/biomarker regions of the present disclosure.

FIG. 4: Sensitivity assessment on probe mix10 showing (A) ΔCt (Group2−Group1) and (B) ΔΔCt (Group2−Group1) with different concentration of spiked foetal DNA. The sample spiked in with trisomy 21 (T21) placenta DNA (mimicking a maternal plasma sample from a woman pregnant with a trisomy 21 foetus) was clearly different from the sample spiked in with normal CVS DNA (mimicking a maternal plasma sample from a woman pregnant with a non-trisomy 21 foetus). Thus, FIG. 4 shows the sensitivity of probes for the detection of biomarker/biomarker regions of the present disclosure.

FIG. 5: Detection of methylated genomic DNA signal in maternal plasma. (A) The figure shows a one-dimensional scatter plot, representing the DNA methylation level in the examined biomarkers. (B) shows another scatter plot, this time depicting the methylation ratio of Group 1 and Group 2 from maternal plasma samples. This comparison visualizes the higher values of methylation ratio of Group 1 and Group 2 in trisomy 21 (T21) samples than in normal samples. Thus, FIG. 5 demonstrates that DNA-methylation level of biomarker/biomarker regions of the present disclosure obtained from trisomy 21 sample is markedly different from DNA-methylation level of samples obtained from non-trisomy 21 sample (normal).

### BRIEF DESCRIPTION OF THE TABLES

Table A shows the classification of different biomarker/biomarker regions as Groups 1 to 4.

Table B shows the DNA methylation of DNA obtained from normal chorionic villus sample versus Trisomy 21 chorionic villus sample or placenta.

Table C shows the DNA methylation of DNA obtained from Trisomy 21 chorionic villus sample or placenta versus normal chorionic villus sample.

Table 1 lists biomarker/biomarker regions that fall within Group 1 as described herein.

Table 2 lists biomarker/biomarker regions that fall within Group 2 as described herein.

Table 3 lists biomarker/biomarker regions that fall within Group 3 as described herein.

Table 4 lists biomarker/biomarker regions that fall within Group 4 as described herein.

Table 5 lists biomarker/biomarker regions that fall within Group 1′ as described herein.

Table 6 lists biomarker/biomarker regions that fall within Group 2′ as described herein.

Table 7 lists biomarker/biomarker regions that fall within Mix10 Group 1 as described herein.

Table 8 lists biomarker/biomarker regions that fall within Mix10 Group 2 as described herein.

### DETAILED DESCRIPTION OF THE PRESENT INVENTION

The inventors of the present disclosure found that depending on the biomarker/biomarker region, the level of DNA-methylation in a foetal DNA and maternal DNA may be different such that the differences may be used to differentiate (1) maternal DNA from foetal DNA and (2) foetal DNA from a foetal with or without the condition or disease. As used herein, the term “disease” and “condition” are interchangeably used to refer to a condition that is not considered to be the norm, normal or healthy. In one example, the disease or condition is Down syndrome or trisomy 21. As used herein, “DNA-methylation” refers to the addition of a methyl group to the cytosine or adenine nucleotides in a DNA sequence. The term “maternal DNA” refers to DNA or polynucleotide obtained from the mother of the foetus or the individual within whose womb the foetus is carried. In one example, the maternal DNA may include, but is not limited to maternal DNA obtained from tissue or cell samples and maternal peripheral blood DNA. In contrast, the term “foetal DNA” refers to DNA or polynucleotide obtained from the foetus or the individual suspected to have the condition or disease.

For example, when maternal blood DNA is close to zero methylation, methylation sensitive enzymes may be used to digest maternal DNA, thus isolating the methylated foetal DNA intact for further analysis. The phrase “zero methylation” means substantially none or about 1%, about 2%, about 3%, about 4%, about 5%, about 6%, about 7%, about 8%, about 9% or about 10% methylation observed. In contrast, when the region in the maternal blood DNA is highly methylated, methylation dependent enzymes can be used to digest maternal DNA to thus isolate the non-methylated foetal DNA intact for further analysis. The phrase “highly methylated” refers to fully, substantially fully or close to 100% methylation or about 100%, about 99%, about 98%, about 97%, about 96%, about 95%, about 94%, about 93%, about 92% or about 90%. Upon removal of maternal DNA by the degree of methylation observed in the maternal DNA, the level of DNA-methylation of the isolated foetal DNA is then analysed. The inventors of the present disclosure found that the isolated foetal DNA from a foetus with a condition or disease would typically be differentially methylated as compared to a foetus without the condition or disease.

Accordingly, disclosed is a method of determining the methylation levels of a biomarker/biomarker region. The method may comprise the steps of: a) treating a sample comprising both foetal and maternal DNA with a reagent that differentially modifies methylated and non-methylated DNA. The method may further comprise b) calculating the percentage of unmodified cytosine residues over the total number of modified and unmodified cytosine residues in order to determine the methylation levels of a biomarker/biomarker region. For example, the reagents may include, but are not limited to sodium bisulfite, one or more enzymes that only cleave methylated DNA, such as methylation dependent enzyme and one or more enzymes that only cleave non-methylated DNA, such as methylation sensitive enzyme. The method of determining the methylation level of biomarker/biomarker region as disclosed herein may further comprise the step of bisulfite sequencing, which may be performed before the step of calculating the percentage of unmodified cytosine residues (i.e. step (b) of the method as described herein). The step of bisulfite sequencing may be a reduced representation bisulfite sequencing (RRBS), which is used to quantify genome wide DNA-methylation profiles in placenta samples from normal individual or individual with the disease or condition. From bisulfite sequencing step, signals detected from the unmodified cytosine residues and the modified cytosine residues are compared to calculate the methylation level.

The method of determining the methylation levels of a biomarker/biomarker region paves the way to an object of the present disclosure of providing a method of screening for biomarker/biomarker regions for Down syndrome. The term “trisomy 21” may be used interchangeably with “Down syndrome” and as used herein refers to a state where an individual or subject or foetus's karyotype is characterized by a complete or partial triplication of human chromosome 21 (HSA21). When an individual or subject or foetus's has partial triplication of human chromosome 21, the individual would be known as a partial trisomy 21. Trisomy 21 leads to complex clinical features and symptoms, for example mental retardation, Alzheimer's disease, seizures, thyroid disorders, cardiac defects, an increased risk of leukaemia, infertility, gastrointestinal defects and early aging.

When the disease or condition is Down syndrome, differentially methylated regions may be selected based on following steps. First, individual CpG sites may be selected. Methylation level of each CpG site may be calculated as:

Methylation level for a CpG=Count of Cytosine/(Count of Cytosine+Count of Thymine)*100%.

Individual CpG sites may be selected using the following criteria:

1) present in at least two normal chorionic villus sample, three T21 chorionic villus sample/placenta, and one maternal blood samples;

2) with difference of average(normal)−average(T21)≧10% or difference of average(T21)−average(normal)≧10%;

3) with a Wilcoxon Rank Sum test p value of ≦0.05.

Next, genomic regions with differential methylation between normal and T21 placenta samples may be selected using the following criteria:

1) at least 2 CpGs (preferrably at least 3 CpGs) with a distance of not more than 150 bp from its nearest neighbor;

2) the average methylation of such regions in maternal blood samples may be either ≧90% or ≦10%;

3) average methylation of such regions in normal samples, named as (average(normal)(region)); and average methylation of such regions in T21 samples named as (average (T21)(region)).

The difference between average (normal)(region) and average (T21)(region) may be at least 10%, except when average maternal blood≦10%, regions with average (normal)(region)≧average(T21)(region) may be also included.

In another example, further selection criteria may be used for more stringent final biomarker selection:
1. For methylation regions with an average(maternal blood)≦10%, regions that may
   1) with average(T21)−average(normal)≧25%; or
   2) average(T21)−average(normal) between 15-25% and ratio of average(T21)/average(normal)≧3; or
   3) average(T21)−average(normal) between 10-15% and ratio of average(T21)/average(normal)≧5 may be selected.
   These regions may be listed as Group 1 biomarkers after an extension of 500-bp both up and downstream for each region.
2. For methylation regions with an average(maternal blood)≦10%, regions that may be:
   1) average(normal)−average(T21)≧10%; or
   2) a subset of regions with average(T21)≦average(normal) were selected. These regions may be listed as Group 2 biomarkers after an extension of 500-bp both up and downstream for each region.
3. For methylation regions with an average(maternal blood)≧90%, regions that have:
   1) average(normal)−average(T21)≧25%, or
   2) (average(normal)−average(T21)) between 15-25% and ratio of (1−average(T21))/(1−average(normal))≧2 were selected.
   These regions may be listed as Group 3 biomarkers after an extension of 500-bp both up and downstream for each region.
4. For methylation regions with an average(maternal blood)≧90%, regions that have:
   1) average(T21)−average(normal)≧25% and ratio of (1−average(normal))/(1−average(T21))>2, or
   2) (average(T21)−average(normal)) between 10-25% and ratio of (1−average(normal))/(1−average(T21))>3 were selected.
   These may be listed as Group 4 biomarkers after an extension of 500-bp both up and downstream for each region.

TABLE A
Classification of different biomarker/biomarker regions as Groups 1 to 4.

| | | DNA methylation | DNA |
|---|---|---|---|
| | Biomarkers falling | level T21 vs. | methylation level |
| Group | within Group | Normal foetus | Maternal DNA |
| Group 1 | Tables 1, 5 and 7 | T21 > Normal | <10% methylation |
| Group 2 | Tables 2, 6 and 8 | T21 < Normal | <10% methylation |
| Group 3 | Table 3 | T21 < Normal | >90% methylation |
| Group 4 | Table 4 | T21 > Normal | >90% methylation |

Tables B and C below list exemplary biomarker/biomarker regions where differences in DNA methylation level may be observed between DNA from maternal blood, DNA from normal sample and DNA from Trisomy 21 sample.

TABLE B
DNA methylation with Normal chorionic villus sample (CVS) >
Trisomy 21 chorionic villus sample/placenta

| | | DNA Methylation level (%) | | | | | |
|---|---|---|---|---|---|---|---|
| | | | | T21 | | | |
| | | Maternal | Normal | CVS/ | Regional | Gene | Functional |
| Chr | Position | Blood | CVS | placenta | Difference | Name | Group |
| chr7 | 6524601- | 97.5 | 57.7 | 21.2 | 36.5 | KDELR2 | Promoter |
| | 6524671 | | | | | | |
| chr17 | 2279396- | 97.0 | 62.6 | 34.2 | 28.4 | SGSM2 | Intragenic |
| | 2279515 | | | | | | |
| chr8 | 92785473- | 97.1 | 82.6 | 54.5 | 28.1 | | Intergenic |
| | 92785570 | | | | | | |
| chr2 | 177042793- | 1.1 | 37.2 | 12.0 | 25.2 | BC047481 | Promoter |
| | 177042924 | | | | | | |
| chr19 | 2621909- | 2.4 | 35.0 | 12.6 | 22.4 | GNG7 | Intragenic |
| | 2621929 | | | | | | |

TABLE C
DNA methylation with Trisomy 21 chorionic villus sample (CVS)/
placenta > Normal chorionic villus sample (CVS)

| | | DNA Methylation level (%) | | | | | |
|---|---|---|---|---|---|---|---|
| | | | | T21 | | | |
| | | Maternal | Normal | CVS/ | Regional | Gene | Functional |
| Chr | Position | Blood | CVS | placenta | Difference | Name | Group |
| chr15 | 89950295- | 3.3 | 18.7 | 59.3 | 40.6 | | Intra- |
| | 89950453 | | | | | | genic |
| chr21 | 38630505- | 1.9 | 47.0 | 82.8 | 35.8 | DSCR3 | Intra- |
| | 38630728 | | | | | | genic |
| chr7 | 49813547- | 2.3 | 5.9 | 40.5 | 34.6 | VWC2 | Pro- |
| | 49813563 | | | | | | moter |
| chr4 | 1189243- | 98.2 | 34.6 | 73.4 | 38.8 | LOC100 | TTS |
| | 1189282 | | | | | 130872 | |
| chr22 | 48391057- | 100.0 | 55.0 | 90.5 | 35.5 | | Intra- |
| | 48391113 | | | | | | genic |

Tables 1 to 8 below list the various biomarker/biomarker regions of the present disclosure. All chromosome coordinates are based on hg19/GRCh37 February 2009 human genome builD002E (which can be accessed at: http://www.ncbi.nlm.nih.gov/assembly/GCF_{—}000001405.13/).

Table 1—The Following Table Shows Group 1 Biomarker/Biomarker Regions

| Chromosome | position ± 500 bp |
|---|---|
| chr20 | 62327556-62328688 |
| chr7 | 156795287-156796392 |
| chr15 | 89949795-89950953 |
| chr16 | 79634775-79635808 |
| chr4 | 90757154-90758285 |
| chr4 | 104640269-104641588 |
| chr2 | 74742958-74744103 |
| chr14 | 103739131-103740259 |
| chr21 | 42797438-42798786 |
| chr10 | 125731845-125733055 |
| chr15 | 101512966-101514272 |
| chr8 | 103135245-103136502 |
| chr13 | 33590446-33591598 |
| chr10 | 109673856-109675121 |
| chr5 | 132158175-132159485 |
| chr7 | 49813047-49814063 |
| chr2 | 74742706-74743768 |
| chr11 | 65779019-65780152 |
| chr5 | 122430786-122431815 |
| chr20 | 62368643-62369892 |
| chr2 | 118943553-118944570 |
| chr8 | 23563507-23564874 |
| chr17 | 36103765-36104809 |
| chr16 | 54323801-54324805 |
| chr15 | 78932964-78933968 |
| chr2 | 214148641-214149777 |
| chr17 | 1011822-1012918 |
| chr7 | 99516170-99517263 |
| chrX | 40125892-40126898 |
| chr2 | 5836739-5837793 |
| chr4 | 90758296-90759398 |
| chr5 | 140011702-140012843 |
| chr1 | 6514210-6515248 |
| chr6 | 5996890-5997909 |
| chr19 | 12305682-12306758 |
| chr4 | 4861330-4862458 |
| chr9 | 135645025-135646255 |
| chr14 | 103673445-103674541 |
| chr7 | 132261359-132262430 |
| chr22 | 26565117-26566148 |
| chr4 | 115519583-115520722 |
| chr4 | 111557910-111559066 |
| chr9 | 66457923-66459013 |
| chr2 | 135475418-135476536 |
| chr4 | 106988-108391 |
| chr14 | 57274628-57275782 |
| chr8 | 25901324-25902546 |
| chr1 | 50890150-50891277 |
| chr11 | 101453003-101454267 |
| chr5 | 178421018-178422525 |
| chr16 | 31548424-31549641 |
| chr8 | 24812205-24813263 |
| chr5 | 92915133-92916248 |
| chr10 | 90342145-90343287 |
| chr1 | 152083001-152084318 |
| chr10 | 27547200-27548767 |
| chr3 | 138663228-138664580 |
| chr19 | 24154030-24155130 |
| chr19 | 23653426-23654731 |
| chr11 | 17741959-17743037 |
| chr6 | 150335417-150336532 |
| chr16 | 67207694-67209016 |
| chr6 | 32632142-32633492 |
| chr17 | 5000103-5001199 |
| chr19 | 51829455-51830465 |
| chr2 | 132181990-132183067 |
| chr13 | 103052783-103053921 |
| chr4 | 85418583-85419780 |
| chr8 | 55366289-55367317 |
| chr20 | 690183-691465 |
| chr10 | 100991951-100993104 |
| chr6 | 88876690-88877844 |
| chr19 | 15343524-15344632 |
| chr1 | 110611097-110612153 |
| chr21 | 36042057-36043111 |
| chr4 | 57521884-57523144 |
| chr5 | 41869248-41870356 |
| chr12 | 114029146-114030172 |
| chr10 | 79397343-79398444 |
| chr3 | 112052013-112053048 |
| chr9 | 74764214-74765324 |
| chrX | 8698540-8699634 |
| chr5 | 92914360-92915699 |
| chr2 | 145273156-145274276 |
| chr12 | 128751594-128752716 |
| chr11 | 91959200-91960279 |
| chr13 | 88324117-88325239 |
| chr13 | 28497927-28499006 |
| chr4 | 11429503-11430522 |
| chr10 | 106399758-106400862 |
| chr2 | 66658936-66659963 |
| chr10 | 124220023-124221086 |
| chr13 | 107186087-107187334 |
| chr1 | 36038365-36039503 |
| chr17 | 35299466-35300667 |
| chr6 | 94128675-94129780 |
| chr7 | 8473676-8474912 |
| chr9 | 971639-972744 |
| chr2 | 163199923-163200927 |
| chr2 | 99553019-99554306 |
| chr6 | 88876067-88877277 |
| chr1 | 65991061-65992095 |
| chr6 | 29855413-29856640 |
| chr5 | 146613621-146614641 |
| chr3 | 124930634-124932388 |
| chr11 | 32354926-32355951 |
| chr13 | 88323676-88324838 |
| chr19 | 15342338-15343517 |
| chr8 | 49782305-49783575 |
| chr3 | 142839524-142840648 |
| chr19 | 2250588-2251672 |
| chr8 | 77592718-77593741 |
| chr13 | 28493763-28495214 |
| chr9 | 87284312-87285413 |
| chrX | 3262935-3264257 |
| chr8 | 70981955-70983203 |
| chr7 | 155249806-155251049 |
| chr7 | 132261841-132262954 |
| chr12 | 3599645-3600720 |
| chr1 | 228224963-228226186 |
| chr7 | 155249472-155250611 |
| chr1 | 197880259-197881268 |
| chr20 | 5296483-5297602 |
| chr14 | 61114773-61116003 |
| chr10 | 22764545-22765835 |
| chr17 | 54990569-54991590 |
| chr4 | 74734622-74735847 |
| chr16 | 31711698-31712811 |
| chr3 | 113251199-113252215 |
| chr2 | 115918734-115919960 |
| chr8 | 21646427-21647429 |
| chr10 | 101088600-101089780 |
| chr1 | 110626620-110627720 |
| chr2 | 115919265-115920294 |
| chr1 | 161228118-161229507 |
| chr9 | 135465566-135466709 |
| chr2 | 171673469-171674701 |
| chr1 | 180203249-180204261 |
| chr16 | 51185313-51186521 |
| chr4 | 6223349-6224451 |
| chr15 | 31775014-31776402 |
| chr8 | 57231942-57233065 |
| chr17 | 75954394-75955543 |
| chr5 | 42951660-42952924 |
| chr7 | 156796321-156797577 |
| chr1 | 24228778-24229812 |
| chr19 | 518649-519755 |
| chr21 | 38630005-38631228 |
| chr9 | 32782722-32783740 |
| chr2 | 172945711-172946769 |
| chr10 | 77156215-77157247 |
| chr3 | 42947042-42948101 |
| chr19 | 12475550-12476612 |
| chr12 | 34260341-34261405 |
| chr14 | 21093532-21094652 |
| chr5 | 177667303-177668321 |
| chr11 | 124790563-124791573 |
| chr8 | 101117982-101119047 |
| chr7 | 156797935-156799045 |
| chr2 | 106681296-106682342 |
| chr2 | 63280742-63281817 |
| chr4 | 87824903-87825959 |
| chr5 | 2748910-2750009 |
| chr13 | 113764818-113765878 |
| chr3 | 111904200-111905251 |
| chr6 | 166073781-166074809 |
| chr5 | 77253364-77254398 |
| chr1 | 75138804-75139849 |
| chr19 | 518371-519433 |
| chr7 | 30721052-30722082 |
| chr2 | 239071774-239072929 |
| chr13 | 112714744-112715860 |
| chr1 | 149672426-149673448 |
| chr1 | 149615701-149616830 |
| chr20 | 61048889-61049961 |
| chr1 | 12227352-12228398 |
| chr15 | 89948892-89949973 |
| chr2 | 223161561-223162697 |
| chr6 | 42694249-42695397 |
| chr11 | 121970597-121971747 |
| chr3 | 138678847-138679945 |
| chr7 | 27141178-27142358 |
| chr10 | 135341690-135342932 |
| chr4 | 154604660-154606098 |
| chr10 | 77166921-77167990 |
| chr7 | 73416687-73417845 |
| chr4 | 13524494-13525631 |
| chr3 | 147114137-147115248 |
| chr2 | 241496948-241498061 |
| chr15 | 98418490-98419500 |
| chr10 | 134600337-134601402 |
| chr9 | 970745-971830 |
| chr4 | 4855901-4856971 |
| chr10 | 128993788-128994821 |
| chr18 | 55103101-55104341 |
| chr6 | 108440069-108441259 |
| chr22 | 20779311-20780375 |
| chr5 | 149339286-149340330 |
| chr18 | 45057772-45058793 |
| chr4 | 56659363-56660374 |
| chr1 | 1475284-1476363 |
| chr6 | 3355941-3357035 |
| chr4 | 174444705-174445862 |
| chr5 | 113391472-113392504 |
| chr19 | 58867314-58868863 |
| chr1 | 110753882-110754909 |
| chr4 | 11369986-11371089 |
| chr19 | 57617794-57618910 |
| chr2 | 19560468-19561882 |
| chr10 | 118893836-118894926 |
| chr2 | 119599365-119600494 |
| chr10 | 102880602-102881746 |
| chr4 | 154710097-154711228 |
| chr10 | 75406913-75407978 |
| chr20 | 21490302-21491310 |
| chr7 | 116963944-116964976 |
| chr21 | 47717342-47718506 |
| chr15 | 76634069-76635191 |
| chr6 | 19836963-19837983 |
| chr14 | 59930967-59932050 |
| chr4 | 4144533-4145643 |
| chr17 | 59529160-59530349 |
| chr1 | 6508439-6509543 |
| chr13 | 44947232-44948683 |
| chr6 | 31782746-31783810 |
| chr9 | 96716921-96717966 |
| chr1 | 36348326-36349400 |
| chr5 | 75377598-75378688 |
| chr19 | 12605940-12607209 |
| chr1 | 47903958-47905010 |
| chrX | 39958047-39959078 |
| chr4 | 90757619-90758716 |
| chr1 | 1475596-1476623 |
| chr19 | 21768903-21770103 |
| chr22 | 24988194-24989219 |
| chr20 | 62119130-62120207 |
| chr13 | 79183133-79184159 |
| chr1 | 110610423-110611546 |
| chr3 | 192231880-192232883 |
| chr1 | 53067432-53068754 |
| chr14 | 101012142-101013156 |
| chr6 | 26383204-26384217 |
| chr1 | 65990519-65991705 |
| chr6 | 159590278-159591499 |
| chr10 | 106402152-106403195 |
| chr5 | 115298082-115299167 |
| chr13 | 28495094-28496169 |
| chr16 | 55090182-55091327 |
| chr2 | 74741146-74742198 |
| chr9 | 972300-973470 |
| chr6 | 58147012-58148096 |
| chr5 | 75378503-75379914 |
| chr10 | 43697466-43698614 |
| chr7 | 64029494-64030615 |
| chr17 | 42733794-42734801 |
| chr1 | 99469575-99470882 |
| chr6 | 29894118-29895181 |
| chr12 | 186616-187663 |
| chr6 | 132271437-132272559 |
| chr16 | 47177934-47179073 |
| chr11 | 31832420-31833516 |
| chr18 | 77557591-77558639 |
| chr6 | 166073966-166074991 |
| chrX | 40034167-40035274 |
| chr6 | 30181343-30182675 |
| chr10 | 22541383-22542500 |
| chr19 | 3585181-3586394 |
| chr10 | 100993393-100994709 |
| chr13 | 79175198-79176772 |
| chr8 | 23145402-23146448 |
| chr13 | 21649464-21650563 |
| chr4 | 75719138-75720217 |
| chr8 | 103136154-103137408 |
| chr1 | 36348827-36350064 |
| chr15 | 28351810-28352993 |
| chr3 | 96533005-96534029 |
| chr16 | 65156230-65157298 |
| chr8 | 55371384-55372441 |
| chr21 | 34443211-34444382 |
| chr5 | 134870249-134871600 |
| chr5 | 77267916-77269224 |
| chr20 | 43726021-43727045 |
| chr16 | 22824422-22825688 |
| chr2 | 115919472-115920484 |
| chr19 | 47742446-47743585 |
| chr20 | 30639826-30640917 |
| chr22 | 51111920-51112989 |
| chr3 | 105085656-105086690 |
| chr14 | 103654845-103656221 |
| chr4 | 190935571-190936589 |
| chr2 | 105473874-105474938 |
| chr11 | 17740927-17742032 |
| chr9 | 139024152-139025261 |
| chr7 | 121945355-121946609 |
| chr7 | 155241820-155243015 |
| chr5 | 140430790-140431893 |
| chr9 | 96712945-96714056 |
| chr6 | 41339962-41341707 |
| chr7 | 156400077-156401426 |
| chr18 | 77159068-77160097 |
| chr18 | 60263106-60264250 |
| chr7 | 19146867-19148026 |
| chr15 | 30516966-30518116 |
| chr1 | 20669276-20670328 |
| chr17 | 70117154-70118558 |
| chrX | 40034885-40036021 |
| chr1 | 114695254-114696299 |
| chr17 | 54911376-54912688 |
| chrX | 9732865-9733965 |
| chrX | 6144580-6145595 |
| chr12 | 31079245-31080319 |
| chr6 | 6003739-6004908 |
| chr1 | 241587181-241588433 |
| chr8 | 41754670-41755694 |
| chr16 | 54964538-54965902 |
| chr4 | 174450805-174451938 |
| chr1 | 159823558-159824677 |
| chr8 | 37823023-37824385 |
| chr5 | 87973673-87974741 |
| chr16 | 67196451-67197670 |
| chr3 | 13114257-13115348 |
| chrX | 114795895-114796907 |
| chr8 | 145924929-145925943 |
| chr4 | 30722888-30724010 |
| chr4 | 1164481-1165501 |
| chr7 | 21984531-21985631 |
| chr6 | 31324194-31325462 |
| chr3 | 45837269-45838527 |
| chr17 | 27942575-27943615 |
| chr11 | 44326881-44327978 |
| chr19 | 8656375-8657381 |
| chr10 | 22765474-22766553 |
| chr18 | 44786948-44788097 |
| chr1 | 33772291-33773475 |
| chr1 | 113286413-113287533 |
| chr7 | 153583712-153584742 |
| chr5 | 82767869-82768964 |
| chr10 | 72217791-72218837 |
| chr12 | 113913247-113914358 |
| chr9 | 140172312-140173550 |
| chr6 | 26501341-26502453 |
| chr17 | 36103118-36104390 |
| chr10 | 42970960-42972143 |
| chr7 | 158936875-158937980 |
| chr2 | 1748371-1749376 |
| chr8 | 69243257-69244288 |
| chr16 | 54970558-54971645 |
| chr8 | 9764031-9765054 |
| chr9 | 842120-843612 |
| chr2 | 172952602-172953626 |
| chr2 | 133427083-133428199 |
| chr14 | 59930491-59931634 |
| chr13 | 20692102-20693181 |
| chr3 | 77088401-77089812 |
| chr18 | 44337399-44338422 |
| chr10 | 126135331-126137188 |
| chr5 | 137577248-137578414 |
| chr1 | 9712287-9713390 |
| chr13 | 79176043-79177076 |
| chr19 | 12605693-12606740 |
| chr14 | 59931412-59932478 |
| chr17 | 66097225-66098410 |
| chr15 | 55581822-55582906 |
| chr17 | 1012309-1013345 |
| chr1 | 53098584-53099635 |
| chr22 | 50241983-50243135 |
| chr12 | 6664496-6665516 |
| chr4 | 186048153-186049274 |
| chr18 | 52988666-52989672 |
| chr17 | 19482933-19483953 |
| chr2 | 225306989-225308020 |
| chr16 | 67449902-67450941 |
| chr15 | 31775691-31776827 |
| chrX | 39955333-39956431 |
| chr11 | 57194026-57195059 |
| chr11 | 111169716-111170774 |
| chr12 | 72665649-72666818 |
| chr1 | 45249552-45250648 |
| chr2 | 29338325-29339442 |
| chr11 | 100997875-100998907 |
| chr7 | 53286535-53287614 |
| chr9 | 132312254-132313324 |
| chr22 | 48971239-48972323 |
| chr2 | 193059493-193060598 |
| chr1 | 10057231-10058257 |
| chr11 | 17742294-17743364 |
| chr11 | 111169255-111170351 |
| chr17 | 75954719-75955800 |
| chr22 | 25816648-25817666 |
| chr2 | 99438484-99439585 |
| chr8 | 41624127-41625221 |
| chr1 | 37499295-37500364 |
| chr2 | 163200364-163201409 |
| chr2 | 107502606-107503922 |
| chr4 | 155664449-155665498 |
| chrX | 40013880-40014965 |
| chrX | 40013390-40014703 |
| chr1 | 114694636-114695802 |
| chrX | 15872020-15873196 |
| chr11 | 30606334-30607434 |
| chr14 | 99740085-99741112 |
| chrX | 39963638-39964797 |
| chr19 | 35454254-35455374 |
| chr13 | 22248958-22249968 |
| chr4 | 187025401-187026581 |
| chr17 | 66192588-66193620 |
| chr2 | 5836246-5837312 |
| chr21 | 34443877-34445016 |
| chr7 | 97360924-97362033 |
| chr1 | 91183001-91184015 |
| chr21 | 27944733-27945766 |
| chr6 | 132271747-132272781 |
| chr2 | 169312310-169313377 |
| chr19 | 59073628-59074697 |
| chr19 | 36821970-36823132 |
| chr14 | 57274109-57275116 |
| chr16 | 88449175-88450191 |
| chr11 | 32460086-32461090 |
| chr1 | 53098242-53099332 |
| chrX | 16729550-16730618 |
| chr6 | 105583839-105584865 |

TABLE 2
Group 2 biomarker/biomarker regions

| Chromosome | position ± 500 bp |
|---|---|
| chr2 | 177042293-177043424 |
| chr17 | 75447059-75448078 |
| chr4 | 7194255-7195262 |
| chr5 | 91589-92757 |
| chr11 | 120856622-120857640 |
| chr19 | 59024982-59026094 |
| chr19 | 940516-941795 |
| chr4 | 7193938-7195038 |
| chr4 | 146654036-146655094 |
| chr11 | 615557-616696 |
| chr15 | 79382604-79383650 |
| chr3 | 196728977-196730077 |
| chr11 | 16631959-16633059 |
| chr16 | 68572564-68573664 |
| chr12 | 124941370-124942436 |
| chr5 | 58335092-58336193 |
| chr5 | 91749-92889 |
| chr7 | 5467057-5468077 |
| chr5 | 139493102-139494173 |
| chr14 | 105662974-105663985 |
| chr8 | 145805846-145806961 |
| chr8 | 79428036-79429146 |
| chr6 | 35265258-35266346 |
| chr16 | 27329710-27330719 |
| chr15 | 45458347-45459411 |
| chr9 | 34989359-34990445 |
| chr7 | 128530459-128531546 |
| chr19 | 52206950-52208110 |
| chr17 | 42430714-42431746 |
| chr16 | 58549538-58550654 |
| chr7 | 104624049-104625214 |
| chr8 | 11659326-11660461 |
| chr9 | 123630409-123631417 |
| chr1 | 201367906-201369041 |
| chr3 | 195633644-195634695 |
| chr18 | 10131461-10132610 |
| chr17 | 43095855-43096977 |
| chr1 | 27667983-27669068 |
| chr2 | 160653729-160654977 |
| chr19 | 7547731-7548834 |
| chr19 | 2621909-2621929 |

TABLE 3
Group 3 biomarker/biomarker regions

| Chromosome | position ± 500 bp |
|---|---|
| chr19 | 1992317-1993338 |
| chr9 | 83918469-83919525 |
| chr8 | 92784973-92786070 |
| chr2 | 131887213-131888412 |
| chr1 | 40349978-40351047 |
| chr2 | 7737860-7738957 |
| chr7 | 6524101-6525171 |
| chr17 | 2278896-2280015 |
| chr5 | 112127163-112128218 |
| chr5 | 107937663-107938712 |
| chr4 | 190867762-190868792 |
| chr15 | 42790858-42791890 |
| chr6 | 25218781-25219919 |
| chrX | 49580354-49581444 |
| chr22 | 46477261-46478350 |
| chr12 | 33042280-33043306 |
| chr14 | 65238928-65240072 |
| chr19 | 13117111-13118120 |
| chr15 | 34446506-34447535 |
| chr13 | 113475775-113476794 |
| chr2 | 88279238-88280344 |
| chr19 | 1214463-1215477 |
| chr16 | 583117-584271 |
| chr16 | 16165071-16166219 |
| chr7 | 75794867-75795908 |
| chr14 | 101528133-101529142 |
| chr12 | 76192585-76193641 |
| chrX | 88311105-88312110 |
| chr6 | 150947074-150948101 |
| chr3 | 51509119-51510233 |
| chr5 | 179003888-179005014 |
| chr16 | 3706134-3707245 |
| chr16 | 585552-586647 |
| chr1 | 245365516-245366599 |
| chr19 | 5632940-5634086 |
| chr17 | 18866459-18867472 |
| chr5 | 149776287-149777294 |
| chr14 | 102446215-102447310 |

TABLE 4
Group 4 biomarker/biomarker regions

| Chromosome | position ± 500 bp |
|---|---|
| chr22 | 48390557-48391613 |
| chr1 | 1563371-1564396 |
| chr17 | 64955445-64956457 |
| chr17 | 25308644-25309779 |
| chr22 | 20711278-20712384 |
| chr11 | 65634906-65635965 |
| chr19 | 36430013-36431127 |
| chr2 | 120753853-120755007 |
| chr3 | 88352344-88353355 |
| chr12 | 118902540-118903563 |
| chr9 | 108281416-108282562 |
| chr5 | 140051482-140052560 |
| chr1 | 160898735-160899806 |
| chr18 | 76572890-76573894 |
| chr4 | 1188743-1189782 |
| chr8 | 63055627-63056660 |
| chr21 | 46408455-46409519 |
| chr17 | 26369562-26370575 |
| chr12 | 34357829-34359018 |
| chr16 | 3422508-3423625 |
| chr11 | 4355415-4356462 |
| chr12 | 96503707-96504816 |
| chr6 | 113010397-113011423 |
| chr14 | 70700394-70701467 |
| chr2 | 127817448-127818633 |
| chr13 | 21833885-21835024 |
| chr1 | 8086820-8087841 |
| chr4 | 1008272-1009402 |
| chr16 | 4164416-4165435 |
| chr9 | 133925782-133926857 |
| chr5 | 71715634-71716664 |
| chr8 | 143376293-143377346 |
| chr9 | 136341686-136342707 |
| chr4 | 125571381-125572445 |
| chr16 | 31499692-31500779 |
| chr22 | 41331407-41332439 |
| chr5 | 1123224-1124264 |
| chr10 | 134944203-134945216 |
| chr7 | 157234758-157235834 |
| chr19 | 1108075-1109206 |
| chr3 | 153827190-153828263 |
| chr2 | 25855948-25857091 |
| chr20 | 30768268-30769293 |
| chr19 | 612209-613468 |
| chr1 | 16810272-16811297 |
| chr10 | 35394872-35395895 |
| chr6 | 33632081-33633160 |
| chr7 | 70502101-70503116 |
| chr19 | 56459039-56460056 |
| chr4 | 126665714-126666723 |
| chr21 | 44145263-44146282 |
| chr22 | 46302758-46303769 |
| chr18 | 77169944-77170972 |
| chr17 | 80138618-80139679 |
| chr11 | 129910118-129911305 |
| chr18 | 74769508-74770618 |
| chr22 | 31050196-31051220 |
| chr18 | 77246168-77247325 |
| chr17 | 54927087-54928213 |
| chr17 | 4352117-4353135 |
| chr20 | 37035846-37036880 |
| chr20 | 58507122-58508294 |
| chr6 | 70176629-70177636 |
| chr2 | 190260853-190261931 |
| chr1 | 1228438-1229528 |
| chr7 | 6193070-6194100 |
| chr8 | 122839743-122840792 |
| chr4 | 48294388-48295416 |
| chr4 | 751860-752999 |
| chr9 | 34038719-34039789 |
| chr6 | 131845507-131846517 |
| chr6 | 149559266-149560360 |
| chr2 | 241826047-241827158 |
| chr8 | 144945405-144946658 |
| chr7 | 150094165-150095247 |
| chr12 | 66282137-66283144 |
| chr19 | 4217153-4218288 |
| chr22 | 50721576-50722871 |
| chr22 | 45808306-45809481 |
| chr15 | 100550723-100551746 |
| chr10 | 131903386-131904407 |
| chr1 | 245427653-245428772 |
| chr7 | 150093627-150094712 |
| chr2 | 182985970-182987017 |
| chr22 | 50721296-50722325 |

TABLE 5
Group 1′ biomarker/biomarker regions

| Chromosome | position ± 500 bp |
|---|---|
| chr20 | 62327556-62328688 |
| chr7 | 156795287-156796392 |
| chr15 | 89949795-89950953 |
| chr16 | 79634775-79635808 |
| chr4 | 90757154-90758285 |
| chr4 | 104640269-104641588 |
| chr2 | 74742958-74744103 |
| chr14 | 103739131-103740259 |
| chr21 | 42797438-42798786 |
| chr10 | 125731845-125733055 |
| chr15 | 101512966-101514272 |
| chr8 | 103135245-103136502 |
| chr13 | 33590446-33591598 |
| chr10 | 109673856-109675121 |
| chr5 | 132158175-132159485 |
| chr2 | 74742706-74743768 |
| chr5 | 122430786-122431815 |
| chr20 | 62368643-62369892 |
| chr2 | 118943553-118944570 |
| chr8 | 23563507-23564874 |
| chr17 | 36103765-36104809 |
| chr16 | 54323801-54324805 |
| chr15 | 78932964-78933968 |
| chr2 | 214148641-214149777 |
| chr17 | 1011822-1012918 |
| chr7 | 99516170-99517263 |
| chrX | 40125892-40126898 |
| chr2 | 5836739-5837793 |
| chr4 | 90758296-90759398 |
| chr5 | 140011702-140012843 |
| chr1 | 6514210-6515248 |
| chr6 | 5996890-5997909 |
| chr19 | 12305682-12306758 |
| chr9 | 135645025-135646255 |
| chr14 | 103673445-103674541 |
| chr7 | 132261359-132262430 |
| chr22 | 26565117-26566148 |
| chr4 | 115519583-115520722 |
| chr9 | 66457923-66459013 |
| chr2 | 135475418-135476536 |
| chr4 | 106988-108391 |
| chr14 | 57274628-57275782 |
| chr8 | 25901324-25902546 |
| chr1 | 50890150-50891277 |
| chr11 | 101453003-101454267 |
| chr16 | 31548424-31549641 |
| chr8 | 24812205-24813263 |
| chr5 | 92915133-92916248 |
| chr10 | 90342145-90343287 |
| chr1 | 152083001-152084318 |
| chr10 | 27547200-27548767 |
| chr3 | 138663228-138664580 |
| chr19 | 24154030-24155130 |
| chr19 | 23653426-23654731 |
| chr11 | 17741959-17743037 |
| chr6 | 150335417-150336532 |
| chr16 | 67207694-67209016 |
| chr6 | 32632142-32633492 |
| chr17 | 5000103-5001199 |
| chr19 | 51829455-51830465 |
| chr2 | 132181990-132183067 |
| chr13 | 103052783-103053921 |
| chr4 | 85418583-85419780 |
| chr8 | 55366289-55367317 |
| chr20 | 690183-691465 |
| chr10 | 100991951-100993104 |
| chr6 | 88876690-8877844 |
| chr19 | 15343524-15344632 |
| chr1 | 110611097-110612153 |
| chr21 | 36042057-36043111 |
| chr4 | 57521884-57523144 |
| chr5 | 41869248-41870356 |
| chr12 | 114029146-114030172 |
| chr10 | 79397343-79398444 |
| chr3 | 112052013-112053048 |
| chr9 | 74764214-74765324 |
| chrX | 8698540-8699634 |
| chr5 | 92914360-92915699 |
| chr2 | 145273156-145274276 |
| chr12 | 128751594-128752716 |
| chr11 | 91959200-91960279 |
| chr13 | 88324117-88325239 |
| chr13 | 28497927-28499006 |
| chr4 | 11429503-11430522 |
| chr10 | 106399758-106400862 |
| chr2 | 66658936-66659963 |
| chr10 | 124220023-124221086 |
| chr13 | 107186087-107187334 |
| chr1 | 36038365-36039503 |
| chr17 | 35299466-35300667 |
| chr7 | 8473676-8474912 |
| chr9 | 971639-972744 |
| chr2 | 163199923-163200927 |
| chr2 | 99553019-99554306 |
| chr6 | 88876067-88877277 |
| chr1 | 65991061-65992095 |
| chr6 | 29855413-29856640 |
| chr5 | 146613621-146614641 |
| chr3 | 124930634-124932388 |
| chr11 | 32354926-32355951 |
| chr13 | 88323676-88324838 |
| chr19 | 15342338-15343517 |
| chr8 | 49782305-49783575 |
| chr3 | 142839524-142840648 |
| chr19 | 2250588-2251672 |
| chr8 | 77592718-77593741 |
| chr13 | 28493763-28495214 |
| chr9 | 87284312-87285413 |
| chrX | 3262935-3264257 |
| chr8 | 70981955-70983203 |
| chr7 | 132261841-132262954 |
| chr12 | 3599645-3600720 |
| chr1 | 228224963-228226186 |
| chr20 | 5296483-5297602 |
| chr14 | 61114773-61116003 |
| chr10 | 22764545-22765835 |
| chr17 | 54990569-54991590 |
| chr4 | 74734622-74735847 |
| chr16 | 31711698-31712811 |
| chr2 | 115918734-115919960 |
| chr8 | 21646427-21647429 |
| chr10 | 101088600-101089780 |
| chr1 | 110626620-110627720 |
| chr2 | 115919265-115920294 |
| chr1 | 161228118-161229507 |
| chr9 | 135465566-135466709 |
| chr1 | 180203249-180204261 |
| chr16 | 51185313-51186521 |
| chr4 | 6223349-6224451 |
| chr15 | 31775014-31776402 |
| chr8 | 57231942-57233065 |
| chr17 | 75954394-75955543 |
| chr5 | 42951660-42952924 |
| chr7 | 156796321-156797577 |
| chr1 | 24228778-24229812 |
| chr19 | 518649-519755 |
| chr21 | 38630005-38631228 |
| chr9 | 32782722-32783740 |
| chr2 | 172945711-172946769 |
| chr10 | 77156215-77157247 |
| chr3 | 42947042-42948101 |
| chr19 | 12475550-12476612 |
| chr12 | 34260341-34261405 |
| chr14 | 21093532-21094652 |
| chr5 | 177667303-177668321 |
| chr11 | 124790563-124791573 |
| chr8 | 101117982-101119047 |
| chr7 | 156797935-156799045 |
| chr2 | 106681296-106682342 |
| chr2 | 63280742-63281817 |
| chr4 | 87824903-87825959 |
| chr5 | 2748910-2750009 |
| chr13 | 113764818-113765878 |
| chr3 | 111904200-111905251 |
| chr6 | 166073781-166074809 |
| chr5 | 77253364-77254398 |
| chr1 | 75138804-75139849 |
| chr19 | 518371-519433 |
| chr7 | 30721052-30722082 |
| chr2 | 239071774-239072929 |
| chr13 | 112714744-112715860 |
| chr1 | 149672426-149673448 |
| chr1 | 149615701-149616830 |
| chr20 | 61048889-61049961 |
| chr15 | 89948892-89949973 |
| chr2 | 223161561-223162697 |
| chr6 | 42694249-42695397 |
| chr11 | 121970597-121971747 |
| chr3 | 138678847-138679945 |
| chr7 | 27141178-27142358 |
| chr10 | 135341690-135342932 |
| chr4 | 154604660-154606098 |
| chr10 | 77166921-77167990 |
| chr7 | 73416687-73417845 |
| chr4 | 13524494-13525631 |
| chr3 | 147114137-147115248 |
| chr2 | 241496948-241498061 |
| chr15 | 98418490-98419500 |
| chr10 | 134600337-134601402 |
| chr9 | 970745-971830 |
| chr4 | 4855901-4856971 |
| chr10 | 128993788-128994821 |
| chr18 | 55103101-55104341 |
| chr6 | 108440069-108441259 |
| chr22 | 20779311-20780375 |
| chr5 | 149339286-149340330 |
| chr18 | 45057772-45058793 |
| chr4 | 56659363-56660374 |
| chr1 | 1475284-1476363 |
| chr6 | 3355941-3357035 |
| chr4 | 174444705-174445862 |
| chr5 | 113391472-113392504 |
| chr19 | 58867314-58868863 |
| chr4 | 11369986-11371089 |
| chr19 | 57617794-57618910 |
| chr2 | 19560468-19561882 |
| chr10 | 118893836-118894926 |
| chr2 | 119599365-119600494 |
| chr10 | 102880602-102881746 |
| chr4 | 154710097-154711228 |
| chr10 | 75406913-75407978 |
| chr20 | 21490302-21491310 |
| chr7 | 116963944-116964976 |
| chr21 | 47717342-47718506 |
| chr15 | 76634069-76635191 |
| chr6 | 19836963-19837983 |
| chr14 | 59930967-59932050 |
| chr4 | 4144533-4145643 |
| chr17 | 59529160-59530349 |
| chr1 | 6508439-6509543 |
| chr13 | 44947232-44948683 |
| chr6 | 31782746-31783810 |
| chr9 | 96716921-96717966 |
| chr1 | 36348326-36349400 |
| chr5 | 75377598-75378688 |
| chr19 | 12605940-12607209 |
| chr1 | 47903958-47905010 |
| chrX | 39958047-39959078 |
| chr4 | 90757619-90758716 |
| chr1 | 1475596-1476623 |
| chr19 | 21768903-21770103 |
| chr22 | 24988194-24989219 |
| chr20 | 62119130-62120207 |
| chr13 | 79183133-79184159 |
| chr1 | 110610423-110611546 |
| chr3 | 192231880-192232883 |
| chr14 | 101012142-101013156 |
| chr6 | 26383204-26384217 |
| chr1 | 65990519-65991705 |
| chr6 | 159590278-159591499 |
| chr10 | 106402152-106403195 |
| chr5 | 115298082-115299167 |
| chr13 | 28495094-28496169 |
| chr16 | 55090182-55091327 |
| chr9 | 972300-973470 |
| chr6 | 58147012-58148096 |
| chr5 | 75378503-75379914 |
| chr10 | 43697466-43698614 |
| chr7 | 64029494-64030615 |
| chr17 | 42733794-42734801 |
| chr1 | 99469575-99470882 |
| chr6 | 29894118-29895181 |
| chr12 | 186616-187663 |
| chr6 | 132271437-132272559 |
| chr16 | 47177934-47179073 |
| chr11 | 31832420-31833516 |
| chr18 | 77557591-77558639 |
| chr6 | 166073966-166074991 |
| chrX | 40034167-40035274 |
| chr6 | 30181343-30182675 |
| chr10 | 22541383-22542500 |
| chr19 | 3585181-3586394 |
| chr10 | 100993393-100994709 |
| chr13 | 79175198-79176772 |
| chr8 | 23145402-23146448 |
| chr13 | 21649464-21650563 |
| chr4 | 75719138-75720217 |
| chr8 | 103136154-103137408 |
| chr1 | 36348827-36350064 |
| chr15 | 28351810-28352993 |
| chr3 | 96533005-96534029 |
| chr16 | 65156230-65157298 |
| chr8 | 55371384-55372441 |
| chr21 | 34443211-34444382 |
| chr5 | 134870249-134871600 |
| chr5 | 77267916-77269224 |
| chr20 | 43726021-43727045 |
| chr16 | 22824422-22825688 |
| chr2 | 115919472-115920484 |
| chr19 | 47742446-47743585 |
| chr20 | 30639826-30640917 |
| chr22 | 51111920-51112989 |
| chr3 | 105085656-105086690 |
| chr14 | 103654845-103656221 |
| chr4 | 190935571-190936589 |
| chr2 | 105473874-105474938 |
| chr11 | 17740927-17742032 |
| chr9 | 139024152-139025261 |
| chr7 | 121945355-121946609 |
| chr7 | 155241820-155243015 |
| chr5 | 140430790-140431893 |
| chr9 | 96712945-96714056 |
| chr6 | 41339962-41341707 |
| chr7 | 156400077-156401426 |
| chr18 | 77159068-77160097 |
| chr18 | 60263106-60264250 |
| chr7 | 19146867-19148026 |
| chr15 | 30516966-30518116 |
| chr1 | 20669276-20670328 |
| chr17 | 70117154-70118558 |
| chrX | 40034885-40036021 |
| chr1 | 114695254-114696299 |
| chrX | 9732865-9733965 |
| chrX | 6144580-6145595 |
| chr12 | 31079245-31080319 |
| chr6 | 6003739-6004908 |
| chr1 | 241587181-241588433 |
| chr8 | 41754670-41755694 |
| chr16 | 54964538-54965902 |
| chr4 | 174450805-174451938 |
| chr1 | 159823558-159824677 |
| chr8 | 37823023-37824385 |
| chr5 | 87973673-87974741 |
| chr16 | 67196451-67197670 |
| chr3 | 13114257-13115348 |
| chrX | 114795895-114796907 |
| chr8 | 145924929-145925943 |
| chr4 | 30722888-30724010 |
| chr4 | 1164481-1165501 |
| chr7 | 21984531-21985631 |
| chr6 | 31324194-31325462 |
| chr17 | 27942575-27943615 |
| chr19 | 8656375-8657381 |
| chr10 | 22765474-22766553 |
| chr18 | 44786948-44788097 |
| chr1 | 33772291-33773475 |
| chr1 | 113286413-113287533 |
| chr7 | 153583712-153584742 |
| chr5 | 82767869-82768964 |
| chr10 | 72217791-72218837 |
| chr12 | 113913247-113914358 |
| chr6 | 26501341-26502453 |
| chr17 | 36103118-36104390 |
| chr10 | 42970960-42972143 |
| chr7 | 158936875-158937980 |
| chr2 | 1748371-1749376 |
| chr8 | 69243257-69244288 |
| chr16 | 54970558-54971645 |
| chr8 | 9764031-9765054 |
| chr9 | 842120-843612 |
| chr2 | 172952602-172953626 |
| chr2 | 133427083-133428199 |
| chr14 | 59930491-59931634 |
| chr13 | 20692102-20693181 |
| chr3 | 77088401-77089812 |
| chr18 | 44337399-44338422 |
| chr10 | 126135331-126137188 |
| chr5 | 137577248-137578414 |
| chr1 | 9712287-9713390 |
| chr13 | 79176043-79177076 |
| chr19 | 12605693-12606740 |
| chr14 | 59931412-59932478 |
| chr17 | 66097225-66098410 |
| chr15 | 55581822-55582906 |
| chr17 | 1012309-1013345 |
| chr1 | 53098584-53099635 |
| chr22 | 50241983-50243135 |
| chr4 | 186048153-186049274 |
| chr18 | 52988666-52989672 |
| chr17 | 19482933-19483953 |
| chr2 | 225306989-225308020 |
| chr16 | 67449902-67450941 |
| chr15 | 31775691-31776827 |
| chrX | 39955333-39956431 |
| chr11 | 57194026-57195059 |
| chr11 | 111169716-111170774 |
| chr12 | 72665649-72666818 |
| chr1 | 45249552-45250648 |
| chr2 | 29338325-29339442 |
| chr11 | 100997875-100998907 |
| chr7 | 53286535-53287614 |
| chr9 | 132312254-132313324 |
| chr22 | 48971239-48972323 |
| chr2 | 193059493-193060598 |
| chr1 | 10057231-10058257 |
| chr11 | 17742294-17743364 |
| chr11 | 111169255-111170351 |
| chr17 | 75954719-75955800 |
| chr22 | 25816648-25817666 |
| chr2 | 99438484-99439585 |
| chr8 | 41624127-41625221 |
| chr1 | 37499295-37500364 |
| chr2 | 163200364-163201409 |
| chr4 | 155664449-155665498 |
| chrX | 40013880-40014965 |
| chrX | 40013390-40014703 |
| chr1 | 114694636-114695802 |
| chrX | 15872020-15873196 |
| chr11 | 30606334-30607434 |
| chr14 | 99740085-99741112 |
| chrX | 39963638-39964797 |
| chr19 | 35454254-35455374 |
| chr13 | 22248958-22249968 |
| chr4 | 187025401-187026581 |
| chr17 | 66192588-66193620 |
| chr2 | 5836246-5837312 |
| chr21 | 34443877-34445016 |
| chr7 | 97360924-97362033 |
| chr1 | 91183001-91184015 |
| chr21 | 27944733-27945766 |
| chr6 | 132271747-132272781 |
| chr2 | 169312310-169313377 |
| chr19 | 59073628-59074697 |
| chr19 | 36821970-36823132 |
| chr14 | 57274109-57275116 |
| chr16 | 88449175-88450191 |
| chr11 | 32460086-32461090 |
| chr1 | 53098242-53099332 |
| chrX | 16729550-16730618 |
| chr6 | 105583839-105584865 |

TABLE 6
Group 2′ biomarker/biomarker regions

| Chromosome | position ± 500 bp |
|---|---|
| chr2 | 177042293-177043424 |
| chr17 | 75447059-75448078 |
| chr4 | 7194255-7195262 |
| chr5 | 91589-92757 |
| chr11 | 120856622-120857640 |
| chr19 | 59024982-59026094 |
| chr19 | 940516-941795 |
| chr4 | 7193938-7195038 |
| chr4 | 146654036-146655094 |
| chr11 | 615557-616696 |
| chr15 | 79382604-79383650 |
| chr3 | 196728977-196730077 |
| chr11 | 16631959-16633059 |
| chr16 | 68572564-68573664 |
| chr12 | 124941370-124942436 |
| chr5 | 58335092-58336193 |
| chr5 | 91749-92889 |
| chr7 | 5467057-5468077 |
| chr5 | 139493102-139494173 |
| chr14 | 105662974-105663985 |
| chr8 | 145805846-145806961 |
| chr8 | 79428036-79429146 |
| chr6 | 35265258-35266346 |
| chr16 | 27329710-27330719 |
| chr15 | 45458347-45459411 |
| chr9 | 34989359-34990445 |
| chr7 | 128530459-128531546 |
| chr19 | 52206950-52208110 |
| chr17 | 42430714-42431746 |
| chr16 | 58549538-58550654 |
| chr7 | 104624049-104625214 |
| chr8 | 11659326-11660461 |
| chr9 | 123630409-123631417 |
| chr1 | 201367906-201369041 |
| chr3 | 195633644-195634695 |
| chr18 | 10131461-10132610 |
| chr17 | 43095855-43096977 |
| chr1 | 27667983-27669068 |
| chr19 | 7547731-7548834 |

TABLE 7
Mix10 Group 1 biomarkers

| Chro- | | | | SEQ | | SEQ |
|---|---|---|---|---|---|---|
| mo- | | Probe | | ID | Second Probe | ID |
| some | position ± 500 bp | set | First Probe sequence (5′→3′) | NO: | sequence (5′→3′) | NO: |
| chr20 | 62327556-62328688 | 1 | GCATGGCTGCTGAGATCGTTCCACAGTATG | 1 | CAGCAGGGCAGGCAGCGCCA | 2 |
| | | | AATCTCTACTCCGCGTACAGCCGGCACCGG | | ACATCGATGCGAACGTGCG | |
| chr7 | 156795287-156796392 | 2 | GCATGGCTGCTGAGATCGTTCCACAGTATGA | 3 | CCTCCAGCAGGCCGCT | 4 |
| | | | ATCTCTCCCGGACCTGCGCCGGCCCCTGCG | | CAGTCGCCGCGGATC | |
| | | | | | GATGCGAACGTGCG | |
| chr15 | 89949795-89950953 | 3 | GCATGGCTGCTGAGATCGTTCCACAGTATGA | 5 | CTTGCGGACTGGGAGCGGGC | 6 |
| | | | ATCTCTGGGACAGAGCGCAGGATCCTCTGCG | | GGATCGATGCGAACGTGCG | |
| chr16 | 79634775-79635808 | 4 | GCATGGCTGCTGAGATCGTTCCACAGTATG | 7 | GGCCGGGCGGCGCCCAGCCC | 8 |
| | | | AATCTCTCCCAGCCTTCTGGGCAGGCGCAT | | TTCGATGCGAACGTGCG | |
| chr4 | 90757154-90758285 | 5 | GCATGGCTGCTGAGATCGTTCCACAGTATG | 9 | TCCCGGAGAAGCAGCCTA | 10 |
| | | | AATCTCTCGCGTTTCCCGGGGAAAAGCGGA | | ATCTCTCAGCCCTTCGAT | |
| | | | | | GCGAACGTGCG | |
| chr4 | 104640269-104641588 | 6 | GCATGGCTGCTGAGATCGTTCCACAGTATG | 11 | GGACTGCAGACCGGTGGCGA | 12 |
| | | | AATCTCTCCGTGGGTGAGTGGGAGGGTCCG | | TGGCCTCGATGCGAACGTGCG | |
| chr2 | 74742958-74744103 | 7 | GCATGGCTGCTGAGATCGTTCCACAGTATG | 13 | GCTTGGGAGCCGGCCGGTGG | 14 |
| | | | AATCTCTGGCCGTGCATCTGCGCAACGCTG | | TGGTCGATGCGAACGTGCG | |
| chr14 | 103739131-103740259 | 8 | GCATGGCTGCTGAGATCGTTCCACAGTATG | 15 | CACACCGGGTCCCCCGCGG | 16 |
| | | | AATCTCTTCCAGCTTCCGCGTACCTGCGCG | | CCTTCGATGCGAACGTGCG | |
| chr14 | 103739131-103740259 | 9 | GCATGGCTGCTGAGATCGTTCCACAGTATG | 17 | TCTGGACCACCCAGGCTTGG | 18 |
| | | | AATCTCTCGCCCCGGAGCGGGCGCGTCCT | | CGAGGTCGATGCGAACGTGCG | |
| chr21 | 42797438-42798786 | 10 | GCATGGCTGCTGAGATCGTTCCACAGTATG | 19 | AGCGCGGTTACTGGGCGC | 20 |
| | | | AATCTCTCCAATGCCCTTCTCCGCGCTCCT | | TGCCCTCGATGCGAACGTGCG | |
| chr21 | 42797438-42798786 | 11 | GCATGGCTGCTGAGATCGTTCCACAGTATG | 21 | GGAGCGCTAGTCTCCGCCACG | 22 |
| | | | AATCTCTGCCCCCGTCGTGCCCGTGCTCC | | AACGTCGATGCGAACGTGCG | |
| chr15 | 101512966-101514272 | 12 | GCATGGCTGCTGAGATCGTTCCACA | 23 | CGCGTTCGTGCCAGGGCAGGT | 24 |
| | | | GTATGAATCTCTCCGGTGT | | CTGTCGATGCGAACGTGCG | |
| | | | CGTCCCCCATCGTTACGCAG | | | |
| chr8 | 103135245-103136502 | 13 | GCATGGCTGCTGAGATCGTTCCACAGTATG | 25 | CTGCCCGGAAAGGCCACAG | 26 |
| | | | AATCTCTGCTGGATCCCGGGCCTGCGGAGT | | GAGGCTCGATGCGAACGTGCG | |
| chr13 | 33590446-33591598 | 14 | GCATGGCTGCTGAGATCGTTCCACAGTATG | 27 | GTAGTAGCGCAGCCCCTCGCG | 28 |
| | | | AATCTCTGCAGCCGCTCCAGCAGGCGCCG | | GTTGGTCGATGCGAACGTGCG | |
| chr10 | 109673856-109675121 | 15 | GCATGGCTGCTGAGATCGTTCCACAGTATG | 29 | CGGCGCGGCGCTCTGGGTC | 30 |
| | | | AATCTCTGTGAGCGCGTTCCTCGGCGGCG | | CTCCTCGATGCGAACGTGCG | |
| chr5 | 132158175-132159485 | 16 | GCATGGCTGCTGAGATCGTTCC | 31 | TGCGCGTCTATTGCGCCCT | 32 |
| | | | ACAGTATGAATCTCTGTGCGA | | GCTGTCGATGCGAACGTGCG | |
| | | | GCACTACCGGTGGAGGAGC | | | |
| chr7 | 49813047-49814063* | 17 | GCATGGCTGCTGAGATCGTCCACAGTATGA | 33 | GCACACCGGGCTAGGGCGTC | 34 |
| | | | ATCTCTACCTGCGCGCTCCGCCTGGCGC | | TCTGGTCGATGCGAACGTGCG | |
| chr11 | 65779019-65780152* | 18 | GCATGGCTGCTGAGATCGTTCCACAGTATG | 35 | CAGCGGGAGGTTGGAACGC | 36 |
| | | | AATCTCTCGACCAGGGCCAGGCCCAGCGC | | GCCATTCGATGCGAACGTGCG | |
| chr8 | 23563507-23564874 | 19 | GCATGGCTGCTGAGATCGTTCCACAGTATG | 37 | AGCTGCCCCGCGGCTTCGCCA | 38 |
| | | | AATCTCTTCCTGCGACTGGAGCGCGAGCGG | | CATCGATGCGAACGTGCG | |
| chr8 | 23563507-23564874 | 20 | GCATGGCTGCTGAGATCGTTCC | 39 | TTCTTCCCGCGCCCGTCGAAT | 40 |
| | | | ACAGTATGAATCTCTGCTCTGC | | CCTCTCGATGCGAACGTGCG | |
| | | | ACCTTCCTCCCCCAGCGCT | | | |
| chr15 | 78932964-78933968 | 21 | GCATGGCTGCTGAGATCGTTCCACAGTAT | 41 | CGCGCGCCTTCCCTGGTCCT | 42 |
| | | | GAATCTCTACCCGGCCCCGCCGGCCATGAGG | | TTTCTCGATGCGAACGTGCG | |
| chr2 | 214148641-214149777 | 22 | GCATGGCTGCTGAGATCGTTCCACAGTATG | 43 | GCGAGATTCCGGCATCTCTCA | 44 |
| | | | AATCTCTCCCAACGGCCCCCGGGAGCTCTC | | CCCCGTCGATGCGAACGTGCG | |
| chr17 | 1011822-1012918 | 23 | GCATGGCTGCTGAGATCGTTCCACAGTATG | 45 | CAGGCCGGGCGCGCGGGTGT | 46 |
| | | | AATCTCTATGCAGTCCCGGGTCGGGAGCCC | | AGATCGATGCGAACGTGCG | |
| chrX | 40125892-40126898 | 24 | GCATGGCTGCTGAGATCGTTCCACAGTATG | 47 | CGGATGCGTCCGCGGCAGAA | 48 |
| | | | AATCTCTTGCGCCGGGCGGCTGCGCGTCC | | GATGTCGATGCGAACGTGCG | |
| chr4 | 90758296-90759398 | 25 | GCATGGCTGCTGAGATCGTTCCACAGTATGA | 49 | TGCGGTGTGAGCCACCTCCC | 50 |
| | | | ATCTCTCGAGGGCAAAGCGCTCTCGGCGG | | GGCGTCGATGCGAACGTGCG | |
| chr5 | 140011702-140012843 | 26 | GCATGGCTGCTGAGATCGTTCCACAGTATG | 51 | CATCGACGCGCTT | 52 |
| | | | AATCTCTATACTGCCGCGGGTCGGCGTCCG | | TAGAAACGGCTCTAGG | |
| | | | | | TTTCGATGCGAACGTGCG | |
| chr1 | 6514210-6515248 | 27 | GCATGGCTGCTGAGATCGTTCCACAGTATG | 53 | CGGCGCCGACAGGGCGGCCGA | 54 |
| | | | AATCTCTCGGCGCGGATCGACGGTGAAGCG | | GATTCGATGCGAACGTGCG | |
| chr9 | 135645025-134646255 | 28 | GCATGGCTGCTGAGATCGTTCCACAGTATGA | 55 | GCCAAGGGCCACCCCTCGG | 56 |
| | | | ATCTCTCGGGCCCGGAGGCCCAGCCCCGC | | CGCGTCGATGCGAACGTGCG | |
| chr8 | 25901324-25902546 | 29 | GCATGGCTGCTGAGATCGTTCCACAGTATG | 57 | CGCGGGAGGAAAGCCGGCTT | 58 |
| | | | AATCTCTAACACCGGCGCTGGCAGTGGCGG | | CCTGTCGATGCGAACGTGCG | |
| chr19 | 23653426-23654731 | 30 | GCATGGCTGCTGAGATCGTTCCACAGTATGA | 59 | CGGCGCAGAACGCGCTGGCCA | 60 |
| | | | ATCTCTCTGCAGGGCGTGGAGACCCCGCC | | GTCGATGCGAACGTGCG | |
| chr20 | 690183-691465 | 31 | GCATGGCTGCTGAGATCGTTCCACAGTATGA | 61 | GCTGCCGTCTCGCACCCCATC | 62 |
| | | | ATCTCTGCGCCCGCAGGCCCGGCCGCCGC | | CGCGCTCGATGCGAACGTGCG | |
| chr21 | 36042057-36043111 | 32 | GCATGGCTGCTGAGATCGTTCCACAGTATG | 63 | GCCCTTCCTGCCGGACCCT | 64 |
| | | | AATCTCTGCGGCTCACCCGAGACCCGGCGC | | CGGCTCGATGCGAACGTGCG | |
| chr13 | 107186087-107187334 | 33 | GCATGGCTGCTGAGATCGTTCCACAGTATG | 65 | CGCCCCGGCTCCAGGGCTCT | 66 |
| | | | AATCTCTGGGTGGCGGCGCGGTGCCAAGG | | GCGTCGATGCGAACGTGCG | |
| chr5 | 42951660-42952924 | 34 | GCATGGCTGCTGAGATCGTTCCACAGTATG | 67 | CACAGGCAGAGTGCCGCG | 68 |
| | | | AATCTCTATTCCCCGGCTTCGCCGGACGC | | GGTCGATGCGAACGTGCG | |
| chr7 | 156796321-156797577 | 35 | GCATGGCTGCTGAGATCGTTCCACAGTATG | 69 | CTGCAGCGGCTCCGGGTTAAT | 70 |
| | | | AATCTCTGCAGCAGCGCTCCACACCGCGG | | CAGCTCGATGCGAACGTGCG | |

TABLE 8
Mix10 Group 2 biomarker/biomarker regions

| Chro- | | | | SEQ | | SEQ |
|---|---|---|---|---|---|---|
| mo- | | Probe | | ID | First Probe sequence | ID |
| some | position ± 500 bp | set | First Probe sequence (5′→3′) | NO: | (5′→3′) | NO: |
| chr2 | 177042293-177043424 | 1 | GCATGGCTGCTGAGATCGTTCCACACATAG | 71 | GCGGAGAGCGCGGAACGAGC | 72 |
| | | | AGTTCTTGCAGCCTGGCCGCTCGCTGAGGC | | GCGTCGATGCGAACGTGCG | |
| chr17 | 75447059-75448078 | 2 | GCATGGCTGCTGAGATCGTTCCACACATA | 73 | GCCCGCCCACCGGGTCACA | 74 |
| | | | GAGTTCTTCTGGACGCGCCGAGAGCCGCG | | TGGTCGATGCGAACGTGCG | |
| chr4 | 7194255-7195262 | 3 | GCATGGCTGCTGAGATCGTTCCACACATA | 75 | TCCGGCCAGCGGCGCCGCCC | 76 |
| | | | GAGTTCTTAGACCTGCGCCCGCGAAGCCAC | | GCTTCGATGCGAACGTGCG | |
| chr5 | 91589-92757 | 4 | GCATGGCTGCTGAGATCGTTCCACACATAG | 77 | GCCCACCTGGGCGGCTCCT | 78 |
| | | | AGTTCTTAGCTCCCGCCTAGCCCCGGACGC | | CCCTCGATGCGAACGTGCG | |
| chr5 | 91589-92757 | 5 | GCATGGCTGCTGAGATCGTTCCACACATAG | 79 | GCGCGTTCCCGGGGCCCCG | 80 |
| | | | AGTTCTTGGCCGAGGCCGCCTTCGCCGC | | CCTCGATGCGAACGTGCG | |
| chr11 | 120856622-120857640 | 6 | GCATGGCTGCTGAGATCGTTCCACACATAG | 81 | GCCCGGTCAGGGAGCAGGG | 82 |
| | | | AGTTCTTCTGAGGCCCCAGCCAGAGACCGC | | TCCATCGATGCGAACGTGCG | |
| chr19 | 59024982-59026094 | 7 | GCATGGCTGCTGAGATCGTTCCACACATAGA | 83 | AGTGCGCCGTCTGCGCCAAGCG | 84 |
| | | | GTTCTTGCACACCGGCGTGCGCGCCTTCC | | CTTCACTCGATGCGAACGTGCG | |
| chr19 | 59024982-59026094 | 8 | GCATGGCTGCTGAGATCGTTCCACACATAG | 85 | CGCATGTGCACGTTGAGCGAGC | 86 |
| | | | AGTTCTTCGCGCGCTCGGGCCGGTGAGTG | | TCTTTCGATGCGAACGTGCG | |
| chr19 | 940516-941795 | 9 | GCATGGCTGCTGAGATCGTTCCACACATAG | 87 | GCAGGGGCCAGATAAGGCTCT | 88 |
| | | | AGTTCTTTGACCGGACGGCCAGGCGGTGGC | | TCCGGCTCGATGCGAACGTGCG | |
| chr4 | 146654036-146655094 | 10 | GCATGGCTGCTGAGATCGTTCCACACATAG | 89 | GCCCCAGGTCATGTGCAGC | 90 |
| | | | AGTTCTTGACGGCAGCGGGCTCCTTCCCGC | | CCCTGTCGATGCGAACGTGCG | |
| chr11 | 615557-616696 | 11 | GCATGGCTGCTGAGATCGTTCCACACATAGA | 91 | GCGGAGTAGGGAGGAGTGGAG | 92 |
| | | | GTTCTTAGCGGAACCCCGCCCCGGCCAGC | | GGCGTCGATGCGAACGTGCG | |
| chr15 | 79382604-79383650 | 12 | GCATGGCTGCTGAGATCGTTCCACACATAG | 93 | CAGATCGCCAGTCCCTCAGTTTG | 94 |
| | | | AGTTCTTCGCCTGCCCCTCGCCAGCGCG | | CCCGGCTCGATGCGAACGTGCG | |
| chr3 | 196728977-196730077 | 13 | GCATGGCTGCTGAGATCGTTCCACACATAG | 95 | CCAGCTCCATCCCGGGCCAG | 96 |
| | | | AGTTCTTCCTCGTCCCCCACTGTGGCCGCG | | CCGCGTCGATGCGAACGTGCG | |
| chr11 | 16631959-16633059 | 14 | GCATGGCTGCTGAGATCGTTCCACACATAG | 97 | CCGGGCGCTGAGCTCCG | 98 |
| | | | AGTTCTTAGCCCCACGCGGCGCAAGAACC | | GGTCGATGCGAACGTGCG | |
| chr16 | 68572564-68573664 | 15 | GCATGGCTGCTGAGATCGTTCCACACATAG | 99 | TCCCAGAGGCCCGCGCGC | 100 |
| | | | AGTTCTTACGCCGGGCGCGCGAACTACACT | | GCAGGTCGATGCGAACGTGCG | |
| chr5 | 58335092-58336193 | 16 | GCATGGCTGCTGAGATCGTTCCACACATAG | 101 | CTCCTCGTCTGCACTTCAAAGCGA | 102 |
| | | | AGTTCTTGACACACACGCTCGCGCCCGCGC | | GTGGCGCTCGATGCGAACGTGCG | |
| chr5 | 91749-92889 | 17 | GCATGGCTGCTGAGATCGTTCCACACATAG | 103 | CGTTCCCGGGGCCCCGCCC | 104 |
| | | | AGTTCTTCCGAGGCCGCGTCGCCGCGCG | | GATCTCGATGCGAACGTGCG | |
| chr5 | 91749-92889 | 18 | GCATGGCTGCTGAGATCGTTCCACACATAG | 105 | GCCTGGGCCGCCCCCGCC | 106 |
| | | | AGTTCTTCCCGTGCCCCCTCTTACCCGGGC | | GTCTTCGATGCGAACGTGCG | |
| chr7 | 5467057-5468077 | 19 | GCATGGCTGCTGAGATCGTTCCACACATAGAG | 107 | TGTATCGCGGCTGGGCCT | 108 |
| | | | TTCTTGGGTTGGCCCGACCTAGACTTGGCGC | | GTCGCATCGATGCGAACGTGCG | |
| chr8 | 145805846-145806961 | 20 | GCATGGCTGCTGAGATCGTTCCACACATAGAG | 109 | GGCGCGGCAGCAGCGTCA | 110 |
| | | | TTCTTCGCCTCGGCGGAGAGCAGCCCCG | | GCCGTTCGATGCGAACGTGCG | |
| chr16 | 27329710-27330719 | 21 | GCATGGCTGCTGAGATCGTTCCACACATAGAG | 111 | AGCGCGTGTTACGTTCAACTTTG | 112 |
| | | | TTCTTCCCCGGGCTGGCACTCGAGATATGTG | | CTTTGCAGTCGATGCGAACGTGCG | |
| chr9 | 34989359-34990445 | 22 | GCATGGCTGCTGAGATCGTTCCACACATAGA | 113 | GGCGCCCTCACCGGTGAGGAG | 114 |
| | | | GTTCTTCCGGGACCCCCGAGTCCTGGCTT | | CTGCTCGATGCGAACGTGCG | |
| chr16 | 58549538-58550654 | 23 | GCATGGCTGCTGAGATCGTTCCACACATAG | 115 | AGCGCCAGCAGCAGTGGCACC | 116 |
| | | | AGTTCTTCCGGGGCCTGCAGCTCGTGGAGC | | CAGCTCGATGCGAACGTGCG | |
| chr8 | 11659326-11660461 | 24 | GCATGGCTGCTGAGATCGTTCCACACATAG | 117 | TCCCAGCCGGGGTAAGCGGAA | 118 |
| | | | AGTTCTTTCCAGTCCCCACAGCGTTCGCGC | | GAAAATCGATGCGAACGTGCG | |
| chr9 | 123630409-123631417 | 25 | GCATGGCTGCTGAGATCGTTCCACACATAGAG | 119 | TCCGCGTGGCATCTAGCACTG | 120 |
| | | | TTCTTACCTCAAGGGGAGAACAGAAGGCCGGC | | TGGAGCTCGATGCGAACGTGCG | |
| chr3 | 195633644-195634695 | 26 | GCATGGCTGCTGAGATCGTTCCACACATAGA | 121 | TGCCACCCAAACACTCATGCACC | 122 |
| | | | GTTCTTTCAGCTTCATCCCATGCCTGTCGCGC | | GGAAGTTCGATGCGAACGTGCG | |

Accordingly, also provided is an isolated biomarker/biomarker region comprising a DNA region of the human genome selected from a DNA region listed in any one of tables 3 to 6 (groups 3, 4, 1′ and 2′). In one example, the isolated biomarker/biomarker region consists of the DNA region of the human genome selected from a DNA region listed in any one of tables 3 to 6 (groups 3, 4, 1′ and 2′). Advantageously, unlike biomarker/biomarker regions known in the art, which are typically obtained from comparison data of DNA obtained from trisomy 21 and normal placenta, the biomarker/biomarker regions of the present disclosure may be used for DNA obtained from bodily fluids.

The term “isolated” as used herein with respect to biomarker/biomarker regions relates to nucleic acids, such as DNA or RNA. In particular, the term “isolated” refers to molecules separated from other DNAs or RNAs, respectively that are present in the natural source of the macromolecule as well as polypeptides. The term “isolated” is meant to include nucleic acid fragments which are not naturally′ occurring as fragments and would not be found in the natural state. For example, the term “isolated” means separated from constituents, cellular and otherwise, in which the cell, tissue, polynucleotide, peptide, polypeptide, protein, antibody or fragment(s) thereof, which are normally associated in nature. An isolated polynucleotide is separated from the 3′ and 5′ contiguous nucleotides with which it is normally associated in its native or natural environment, e.g., on the chromosome.

As used herein, the term “biomarker” or “biomarker region” refer to molecular indicators of a specific biological property, a biochemical feature or facet that can be used to determine the presence or absence and/or severity of a particular disease or condition. As used herein, the term “biomarker” or “biomarker regions” refers to polynucleotide or DNA region whose presence may be associated to a disease or condition. The biomarkers may be differentially present (i.e. partially, complete and/or otherwise present) in a foetus with the disease or condition, the presence of one or more of which can be used to distinguish foetus with an increased risk of the disease or condition and foetus that do not have an increased risk of the disease or condition.

The inventors of the present disclosure found that the biomarker/biomarker region as provided in the present disclosure is identified to be related to Down syndrome or trisomy 21. Thus, in another aspect, there is provided an isolated biomarker/biomarker region for detecting trisomy 21 or partial trisomy 21. The isolated biomarker/biomarker region comprising a DNA region of the human genome selected from a DNA region listed in any one of tables 3 to 6 (groups 3, 4, 1′ and 2′). In one example, the isolated biomarker/biomarker region consists of the DNA region of the human genome selected from a DNA region listed in any one of tables 3 to 6 (groups 3, 4, 1′ and 2′).

The term “partial” as used herein refers to partial triplication of chromosome 21. That is, the extra copy of chromosome 21 is not complete, incomplete or existing only in part.

One way of determining whether the isolated biomarker/biomarker region is related to a disease or condition present in the foetus is by observing the presence of abnormalities or differences as compared to a control sample. For example, the isolated biomarker/biomarker region may have increased or decreased DNA-methylation or DNA mutations such as deletion, frame-shift, insertion, missense, nonsense, point, silent, splice site or translocation.

Thus, in one example, the level of DNA-methylation of any one of the biomarker/biomarker regions in a diseased sample is different from the level of DNA-methylation in the same biomarker/biomarker region of a non-diseased control DNA. The level of DNA-methylation differences may be observed in any one, two, three, four, five, six, seven, eight, nine, 10, 11, 12, 13, 14, 15, 16, 17, 18, 19, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395 or all of the biomarker/biomarker regions of a non-diseased control DNA.

The “non-diseased control DNA” or “negative control” refers to DNA or sample from an individual or a group of individual who do not have the condition or disease and/or who are not carrying a diseased foetus or a foetus with a condition. For example, the “non-diseased control DNA” may include DNA or sample obtained from an individual or group of individual who do not have trisomy 21 or not carrying trisomy 21- or Down syndrome-foetus.

As used herein, the term “different” refers to not the same as the level of DNA-methylation as observed in a non-diseased control DNA. For example, the level of DNA-methylation isolated biomarker/biomarker region may be less or more than a non-diseased control DNA.

In one example, the isolated biomarker/biomarker region referred to in Table 5 (group 1′) or 6 (group 2′) may be methylated at a level less than about 10% in maternal DNA. Thus, the isolated biomarker/biomarker region referred to in Table 5 (group 1′) or 6 (group 2′) may be methylated at a level less than about 9%, less than about 8%, less than about 7%, less than about 6%, less than about 5%, less than about 4%, less than about 3%, less than about 2% or less than about 1% in maternal DNA. That is, the isolated biomarker/biomarker region of a diseased or individual with the condition may be methylated at a level of less than about 10% of the DNA-methylation observed in maternal DNA.

In another example, the isolated biomarker/biomarker region referred to in Table 3 (group 3) or 4 (group 4) may be methylated at a level more than about 90% in maternal DNA. Thus, the isolated biomarker/biomarker region referred to in Table 3 (group 3) or 4 (group 4) is methylated at a level more than about 91%, more than about 92%, more than about 93%, more than about 94%, more than about 95%, more than about 96%, more than about 97%, more than about 98%, more than about 99% or about 100% in maternal DNA. That is, the isolated biomarker/biomarker region of a diseased or individual with the condition may be methylated at a level of more than about 90% of the DNA-methylation observed in maternal DNA.

In one example, the level of the DNA-methylation of the biomarker referred to in Tables 5 (group 1′) or 4 (group 4) in a diseased sample may be higher than the level of DNA-methylation in the same region of a non-diseased control DNA. As used herein, the term “higher” refers to the level of the DNA-methylation to be at least about 10%, at least about 11%, at least about 12%, at least about 13%, at least about 14%, at least about 15%, at least about 16%, at least about 17%, at least about 18%, at least about 19%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95% or at least about 100% higher than the level of DNA-methylation in the same region of a non-diseased control DNA.

On the other hand, the level of DNA-methylation of the biomarker referred to in Tables 6 (group 2′) or 3 (group 3) in a diseased sample may be lower than the level of DNA-methylation in the same region of a non-diseased control DNA. The term “lower” refers to the level of the DNA-methylation to be at least about 1%, at least about 5%, at least about 10%, at least about 11%, at least about 12%, at least about 13%, at least about 14%, at least about 15%, at least about 16%, at least about 17%, at least about 18%, at least about 19%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95% or at least about 100% lower than the level of DNA-methylation in the same region of a non-diseased control DNA.

The present disclosure also provides for an isolated biomarker/biomarker region comprising a DNA region of the human genome selected from a DNA region listed in any one of tables 1 to 4 and 7 to 8 (groups 1 to 4 and Mix10 Group 1 and Mix10 Group 2 respectively). In one example, the isolated biomarker/biomarker region consists of the DNA region of the human genome selected from the DNA region listed in any one of tables 1 to 4 and 7 to 8 (groups 1 to 4 and Mix10 Group 1 and Mix10 Group 2 respectively). The isolated biomarker/biomarker region may be selected from any two, three, four, five or all of tables 1 to 4 and 7 to 8 (groups 1 to 4 and Mix10 Group 1 and Mix10 Group 2 respectively).

The level of DNA-methylation of any one of the biomarker/biomarker regions of tables 1 to 4 and 7 to 8 (groups 1 to 4 and Mix10 Group 1 and Mix10 Group 2 respectively) in a diseased sample may be different from the level of DNA-methylation in the same biomarker/biomarker region of a non-diseased control DNA.

In one example, the isolated biomarker/biomarker region referred to in Tables 1 to 2 and 7 to 8 (Group 1, Group 2, Mix 10 Group 1 and Mix 10 Group 2 respectively) may be methylated at a level less than about 10% in maternal DNA. Thus, the isolated biomarker/biomarker region referred to in Tables 1 to 2 and 7 to 8 (Group 1, Group 2, Mix 10 Group 1 and Mix 10 Group 2 respectively) may be methylated at a level less than about 9%, less than about 8%, less than about 7%, less than about 6%, less than about 5%, less than about 4%, less than about 3%, less than about 2% or less than about 1% in maternal DNA. That is, the isolated biomarker/biomarker region of a diseased or individual with the condition may be methylated at a level of less than about 10% of the DNA-methylation observed in maternal DNA.

In another example, the isolated biomarker/biomarker region referred to in Table 3 (group 3) or 4 (group 4) may be methylated at a level more than about 90% in maternal DNA. Thus, the isolated biomarker/biomarker region referred to in Table 3 (group 3) or 4 (group 4) is methylated at a level more than about 91%, more than about 92%, more than about 93%, more than about 94%, more than about 95%, more than about 96%, more than about 97%, more than about 98%, more than about 99% or about 100% in maternal DNA. That is, the isolated biomarker/biomarker region of a diseased or individual with the condition may be methylated at a level of more than about 90% of the DNA-methylation observed in maternal DNA.

In one example, the level of the DNA-methylation of the biomarker referred to in Table 1 (group 1) or Table 4 (group 4) or Table 7 (Mix10 Group 1) in a diseased sample may be higher than the level of DNA-methylation in the same region of a non-diseased control DNA. As used herein, the term “higher” refers to the level of the DNA-methylation to be at least about 10%, at least about 11%, at least about 12%, at least about 13%, at least about 14%, at least about 15%, at least about 16%, at least about 17%, at least about 18%, at least about 19%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95% or at least about 100% higher than the level of DNA-methylation in the same region of a non-diseased control DNA.

On the other hand, the level of DNA-methylation of the biomarker referred to in Table 2 (group 2) or Table 3 (group 3) or Table 8 (Mix 10 Group 2) in a diseased sample may be lower than the level of DNA-methylation in the same region of a non-diseased control DNA. The term “lower” refers to the level of the DNA-methylation to be at least about 1%, at least about 5%, at least about 10%, at least about 11%, at least about 12%, at least about 13%, at least about 14%, at least about 15%, at least about 16%, at least about 17%, at least about 18%, at least about 19%, or at least about 20%, at least about 25%, at least about 30%, at least about 35% of at least about 40%, at least about 45%, at least about 50%, at least about 55%, at least about 60%, at least about 65%, at least about 70%, at least about 75%, at least about 80%, at least about 85%, at least about 90%, at least about 95% or at least about 100% lower than the level of DNA-methylation in the same region of a non-diseased control DNA.

As the isolated biomarker/biomarker region as described herein are related to a specific disease or condition, it was found that they may be used in the screening of a specific disease or condition in a foetus. Thus, in yet another aspect of the present disclosure there is provided a method determining the likelihood of a foetus to suffer from a specific disease. The method comprising the steps of: a) providing an isolated total DNA sample from a pregnant woman, comprising foetal DNA and maternal DNA. Further comprising the steps of b) removing maternal DNA background; c) measuring a signal indicative for the level of foetal DNA based on one or more biomarkers/biomarker regions, where in the case where the maternal DNA background had a level of methylation below 10%, the signal is the level of methylated foetal DNA and in the case where the maternal DNA background had a level of methylation above 90%, the signal is the level of unmethylated foetal DNA; d) determining a ratio of signals obtained under step c) by dividing the signals of one or more of Group 1 and/or Group 3 biomarkers/biomarker regions over the signals of one or more of Group 2 and/or Group 4 biomarkers/biomarker regions, wherein a ratio higher than the ratio determined in control foetal DNA obtained from a non-diseased foetus indicates that the foetus is likely to suffer from the specific disease.

In one example, each of the groups is characterized by:

Group 1: maternal DNA background has a level of methylation below 10% and the signal of the biomarker/biomarker region is higher in foetal DNA obtained from a foetus suffering from the specific disease compared to the same biomarker/biomarker region in control foetal DNA obtained from a foetus not suffering from the disease.

Group 2: maternal DNA background has a level of methylation below 10% and the signal of the biomarker/biomarker region is lower in foetal DNA obtained from a foetus suffering from the specific disease compared to the same biomarker/biomarker region in control foetal DNA obtained from a foetus not suffering from the disease.

Group 3: maternal DNA background has a level of methylation above 90% and the signal of the biomarker/biomarker region is higher in foetal DNA obtained from a foetus suffering from the specific disease compared to the same biomarker/biomarker region in control foetal DNA obtained from a foetus not suffering from the disease.

Group 4: maternal DNA background has a level of methylation above 90% and the signal of the biomarker/biomarker region is lower in foetal DNA obtained from a foetus suffering from the specific disease compared to the same biomarker/biomarker region in control foetal DNA obtained from a foetus not suffering from the disease.

Specific diseases that may be screened using the method as described herein include Trisomy 18, 13, X and Y and other diseases associated with placenta such as preterm labour, pre-eclampsia and/or eclampsia, intrauterine growth restriction (IUGR), congenital heart diseases. It would be appreciated by the person skilled in the art that for each of these specific diseases, the biomarker/biomarker regions would be those known to be related to the individual specific disease.

In particular, the present disclosure found a method of screening for Down syndrome. Thus, in yet another aspect there is provided a method of determining the likelihood of a foetus to suffer from trisomy 21 or partial trisomy 21. The method comprising the steps of: a) providing an isolated total DNA sample from a pregnant woman, comprising foetal DNA and maternal DNA; b) removing maternal DNA background; c) measuring a signal indicative for the level of foetal DNA based on one or more biomarkers/biomarker regions listed in any one of Tables 1 to 8, where in the case where the maternal DNA background had a level of methylation below 10%, the signal is the level of methylated foetal DNA and in the case where the maternal DNA background had a level of methylation above 90%, the signal is the level of unmethylated foetal DNA; d) determining a ratio of signals obtained under step c) by dividing the signals of one or more of Group 1 and/or Group 3 biomarkers/biomarker regions over the signals of one or more of Group 2 and/or Group 4 biomarkers/biomarker regions, wherein a ratio higher than the ratio determined in control foetal DNA obtained from a non-diseased foetus indicates that the foetus is likely to suffer from trisomy 21 or partial trisomy 21.

In one example, each of the groups is characterized by:

Group 1: biomarker/biomarker region listed in Table 1 (Group 1), Table 5 (Group 1′), or Table 7 (Mix10 Group 1).

Group 2: biomarker/biomarker region listed in Table 2 (Group 2) or Table 6 (Group 2′), or Table 8 (Mix10 Group 2).

Group 3: biomarker/biomarker region listed in Table 3 (Group 3).

Group 4: biomarker/biomarker region listed in Table 4 (Group 4).

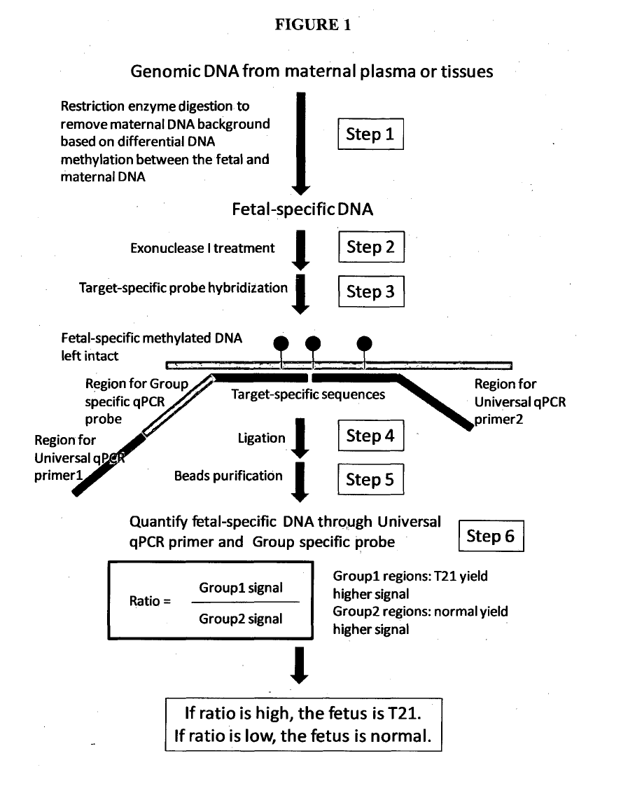

One example of the method of determining the likelihood of a foetus to suffer from a specific disease such as Down syndrome is illustrated in FIG. 1. The method may comprise the steps as described herein. In brief, total genomic DNA obtained from a sample, which may comprise both maternal and foetal DNA, may be processed to remove maternal DNA. The removal of maternal DNA, which may be performed by restriction enzyme digestion based on the methylation status of the maternal DNA, ensure only foetal-specific DNA is analysed in the method as described herein. Once foetal-specific DNA is substantially free of maternal DNA, at least one of the specific region (such as the biomarker/biomarker region as disclosed herein) of the foetal-specific DNA may then be analysed for its signal by using methods known in the art, such as by using detectable label or quantitatively using quantitative polymerase chain reaction (qPCR). When qPCR is used, probes required may include, but are not limited to probes to specific region of a particular group (e.g. region for Group 1, Group 2, Group 3 or Group 4 as described herein), a first universal probe for detection of foetal-specific DNA region and a second universal probe for detection of foetal-specific DNA region. To facilitate with the analysis of foetal-specific DNA after the isolation from maternal DNA, the enzyme digested DNA may be treated with an enzyme that can catalyses the removal of nucleotides from single-stranded DNA in 3′ to 5′ direction and/or to facilitate the removal of the 3′ overhang of the enzyme digested DNA, for example Exonuclease I. The method may also further comprise steps required prior to qPCR, such as target-specific probe hybridization, ligation and beads purification. The ratio of signals obtained from two or more different groups may be calculated and if the ratio is high, the foetus is considered to have trisomy 21. If the ratio is low, the foetus is considered to not have trisomy 21 (i.e. normal).

In the present disclosure, the isolated total DNA may be obtained from biological sample such as, but not limited to biological fluid, cell or tissue sample obtained from an individual suspected of having the disease or condition or the pregnant woman, which can be assayed for biomarkers. For example, the isolated total DNA from step a) in the methods as described herein may be obtained from bodily fluid, tissue sample obtained from the pregnant woman and the like. The “bodily fluid” as used herein refers to any biological fluid, which can comprise cells or be substantially cell free, which can be assayed for biomarkers, including, but is not limited to whole blood, tears, sweat, vaginal secretion, saliva, urine and amniotic fluid. As used herein, whole blood may include, but is not limited to blood cells, plasma and serum. That is, the total DNA as used in the methods of the present disclosure may be obtained from plasma or serum, or the like.

In another example, the isolated total DNA may be obtained from tissue sample obtained from the pregnant woman. In which case, the tissues may include, but are not limited to placental tissue and amniotic sac tissue.

As used herein, when the biological sample is obtained from a pregnant individual, the sample may be obtained in the first, second or third trimester of pregnancy. The term “first trimester” as used herein refers to the period of time within the first third of a pregnant individual's gestation. For example, the “first trimester” can be the period of time within the first three months, the first 12 weeks or about the first 90 days of gestation, for example human gestation. The term “second trimester” as used herein refers to the period of time within the second third of a pregnant individual's gestation. For example, the “second trimester” comprises the period of time within the fourth through sixth months, 13th through 27th weeks, or about days 91 to 180 of gestation, for example human gestation. The term “third trimester” as used herein refers to the period of time within the third or last third of a pregnant individual's gestation. For example, the “third trimester” comprises the period of time within the seventh months through ninth months, 28^{th} weeks through 41^{st} weeks, or about days 181 to 270 of gestation, for example human gestation. Accordingly, as used in the methods as disclosed herein, the maternal DNA may include, but is not limited to maternal DNA obtained from tissue or cell samples and maternal peripheral blood DNA.

To ensure accurate execution of the method of the present disclosure, it is important to remove the maternal DNA from the isolated total DNA. As used herein, the phrase “removing maternal DNA background” refers to partial or full removal of DNA that is not from the foetus or individual suspected to have the condition or disease. The removal of maternal DNA background may lead to substantially no maternal DNA present. As mentioned above, the inventors of the present disclosure discovers that the level of DNA-methylation on the DNA of a foetus and the maternal DNA may be different at different sites. Thus, when the maternal DNA background has a level of methylation below 10%, 9%, 8%, 7%, 6%, 5%, 4%, 3%, 2% or 1%, the signal as measured in the methods of the present disclosure may be the level of methylated foetal DNA. On the other hand, if the maternal DNA background has a level of methylation above 90%, 91%, 92%, 93%, 94%, 95%, 96%, 97%, 98% or 99%, the signal as measured in the methods of the present disclosure may be the level of unmethylated foetal DNA.

The difference in DNA-methylation level in maternal DNA and foetal DNA may be utilised in the step of removing maternal DNA background of the method of the present disclosure. For example, the step of removing maternal DNA background may be performed by treating the total isolated DNA with a reagent that differentially modifies methylated or non-methylated DNA, such as by treating total isolated DNA with an antibody or a protein that can specifically binds to methylated cytosine. For example, the reagents may include, but are not limited to sodium bisulfite, one or more enzymes that only cleave methylated DNA, such as methylation dependent enzyme and one or more enzymes that only cleave non-methylated DNA, such as methylation sensitive enzyme. The enzymes may include, but are not limited to MspJI, LpnPI, FspEI, DpnI, DpnII, McrBC, MspI, HapII, AatII, AciI, AclI, AfeI, AgeI, AscI, AscI, AsiSI, AvaI, BceAI, BmgBI, BsaAI, BsaHI, BsiEI, BsiWI, BsmBI, BspDI, BsrFI, BssHII, BstBI, BstUI, Clal, EagI, FauI, FseI, FspI, HaeII, HgaI, HhaI, HinP1I, HpaII, Hpy99I, HpyCH4IV, KsaI, MluI, NaeI, NarI, NgoMIV, NotI, NruI, Nt.BsmAI, NtCviPII, PaeR7I, PmlI, PvuI, RsrII, SacII, SalI, SfoI, SgrAI, SmaI, TspMI, ZraI and the like.

As known in the art, prior to measuring the signal indicative for the level of foetal DNA based on one or more biomarkers/biomarker regions, the total DNA may be treated with an enzyme which catalyses the removal of nucleotides from single-stranded DNA in the 3′ to 5′direction, for example enzymes such as, but are not limited to exonucleases such as Exonuclease I. This step ensures the removal of the 3′ overhang of a digested DNA. For avoidance of doubt, the 3′ end of a single strand DNA refers to the terminating or tail end of DNA strand which is characterised by the hydroxyl group of the third carbon in the sugar-ring; the 5′ end of a single-strand DNA refers to the end of the DNA that has the fifth carbon in the sugar-ring of the deoxyribose or ribose at its terminus.

To facilitate the detection of biomarker/biomarker regions, the method of the present disclosure may require the addition of one or more probe sets. Thus, in one example, the total DNA of the method of the present disclosure may be incubated with one or more probe sets. In one example, the total DNA may be incubated with one or two or three or four or five or six or seven or eight or nine or ten or 50 or 100 or 200 or 300 or 400 or 500 or 600 or 700 or 800 or 900 or 1000 or 2000 or in order of thousands or more probe sets.

The first probe may include, but is not limited to a sequence for binding a forward primer, a sequence for binding a third probe and a sequence for binding to the one or more biomarker/biomarker regions. The first probe, which binds to a third probe may include, but is not limited to TaqMan® probe or the like. The sequences of the first probe in a probe set may be selected from any one of the probe sets listed in Tables 7 or 8. That is, the first probe in a probe set may be include any one, two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34 or all of the probes listed in Table 7 and/or may include one, two, three, four, five, six, seven, eight, nine, 10, 11, 12, 13, 14, 15, 16, 17, 18, 19, 20, 21, 22, 23, 24, 25 or all of the probes listed in Table 8.

The second probe may include, but is not limited to a sequence for binding a reverse primer and sequence for binding to one or more biomarker/biomarker regions. The second probe may be phosphorylated at the 5′ end. The second probe may include further modification, which allows the probe to be isolated by affinity purification. Such modification may include, but not limited to a 3′ Biotin-TEG modification, which allows the probe to be isolated by bead purification. The sequences of the second probe in a probe set may be selected from any one of the probe sets listed in Tables 7 or 8. That is, the second probe in a probe set may be include any one, two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34 or all of the probes listed in Table 7 and/or may include one, two, three, four, five, six, seven, eight, nine, 10, 11, 12, 13, 14, 15, 16, 17, 18, 19, 20, 21, 22, 23, 24, 25 or all of the probes listed in Table 8.

In view of the above, the sequences of the first probe and second probe in each probe set may be selected from any one of the probe sets listed in Tables 7 and/or 8. When one probe is selected from the probe sets listed in Tables 7 and/or 8, the first probe and second probe from each probe set may be ligated together. That is, the two probes from each probe set may be ligated together. If two or more probes are ligated together, any excess probes which have not been ligated may be removed. Thus, the method of the present disclosure further comprises the step of removing the excess probes which have not been ligated together. The step of removing the excess probes may be performed using bead purification, such as but is not limited to streptavidin beads.

The third probe may include binding sequences that is different for each of biomarker/biomarker region groups 1 to 4. That is, the binding sequence for third probe for the Group 1 biomarker/biomarker region may comprise or consists of the sequence 5′-CCACAGTATGAATCTCT-3′ (SEQ ID NO: 123). For Group 2 biomarker/biomarker region, the binding sequence for third probe may comprise or consists of the sequence 5′-CCACACATAGAGTTCTT-3′ (SEQ ID NO: 124). In one example, the third probe may comprise or consists of the sequence 5′-FAM-CCACAGTATGAATCTCT-MGB-3′ (SEQ ID NO: 125), which is suitable for Mix10 Group 1. In another example, the third probe may comprise or consists of the sequence 5′-VIC-CCACACATAGAGTTCTT-MGB-3′ (SEQ ID NO: 126), which is suitable for Mix 10 Group 2.

In any of the methods of the present disclosure, the signal indicative of the level of foetal DNA may be measured using a detectable label, such as a fluorophore, radioactive moiety, enzyme, biotin/avidin label, chromophore, chemiluminescent label, or the like. Thus, in one example, the signal which is indicative of the level of foetal DNA in step (c) of the methods as described herein may be a fluorescent signal. Different fluorescent signals may be provided and measured for each of biomarker/biomarker region groups 1 to 4. When fluorescent signals are used to detect the level of foetal DNA, the signal would originate from one or more probes having fluorophores thereon.

In an alternative example, the signal indicative of the level of foetal DNA may be measured quantitatively. For example, the signal which is indicative of the level of foetal DNA in step (c) of the methods as described herein may be measured by quantitative polymerase chain reaction.

To facilitate detection, probes of the present disclosure may further comprise forward primer and reverse primers. In one example, the forward primer may comprise or consists of the sequence 5′-GCATGGCTGCTGAGATCGT-3′(SEQ ID NO: 127). The reverse primer may comprise or consists of the sequence 5′-CGCACGTTCGCATCGA-3′(SEQ ID NO: 128).

In one example, the probe set may comprise 5′-FAM-CGGCTGCCACCCG-MGB-3′(SEQ ID NO: 129), which is a specific probe suitable for Group 1, 5′-VIC-CGCGCCTTCCAGTG-MGB-3′(SEQ ID NO: 130), which is a specific probe suitable for Group 2, 5′-ACCCCACAGCGGAGCTC-3′(SEQ ID NO: 131), which is a forward primer suitable for Group 1 and 5′-AACACATGGTCACGCACACC-3′(SEQ ID NO: 132), which is a forward primer suitable for Group 2, 5′-AGAAAAGGACCAGGGAAGGC-3′(SEQ ID NO: 133), which is a reverse primer suitable for group 1 and 5′-CGCTTGGCGCAGACG-3′(SEQ ID NO: 134), which is a reverse primer suitable for group 2.

The present disclosure also contemplates a variety of kits for use in the disclosed methods. For example, there is provided a kit comprising primers for amplifying the one or more biomarkers/biomarker regions selected from any one of the DNA regions of the human genome listed in any one of tables 3 to 6 (groups 3, 4, 1′ and 2′). The kit may further comprise one or more reagents for measuring a signal indicative for the level of foetal DNA based on the one or more biomarkers/biomarker regions.

In yet another example there is provided a kit comprising primers for amplifying the one or more biomarkers/biomarker regions selected from any one of the DNA regions of the human genome listed in any one of tables 1 to 4 (groups 1 to 4). The kit may further comprise one or more reagents for measuring a signal indicative for the level of foetal DNA based on the one or more biomarkers/biomarker regions.

In yet another example there is provided a kit comprising primers for amplifying the one or more biomarkers/biomarker regions selected from any one of the DNA regions of the human genome listed in any one of tables 7 to 8 (Mix10 Group 1 and Mix10 Group 2). The kit may further comprise one or more reagents for measuring a signal indicative for the level of foetal DNA based on the one or more biomarkers/biomarker regions.

The reagents that are suitable for measuring a signal may include reagents that may incorporate a detectable label, such as a fluorophore, radioactive moiety, enzyme, biotin/avidin label, chromophore, chemiluminescent label, or the like, or the kits may include reagents for labeling the nucleic acid primers, the nucleic acid probes or the nucleic acid primers and nucleic acid probes for detecting the presence or absence of the biomarker/biomarker region as described herein. The primers and/or probes, calibrators and/or controls can be provided in separate containers or pre-dispensed into an appropriate assay format, for example, into microtiter plates. The kit may further comprises reagents including, but are not limited to reagents for isolating DNA from samples, reagents for differentially modifying methylated or non-methylated DNA, reagents for polymerase chain reaction and reagents for quantitative polymerase chain reaction. For example, the kits may include reagents used in the Experimental sections below, in particular Example 2 and Example 3.

The kit may further comprise instructions that may be provided in paper form or in computer-readable form, such as a disc, CD, DVD or the like. The kits may optionally include quality control reagents, such as sensitivity panels, calibrators, and positive controls.

The kits can optionally include other reagents required to conduct a diagnostic assay or facilitate quality control evaluations, such as buffers, salts, enzymes, enzyme co-factors, substrates, detection reagents, and the like. Other components, such as buffers and solutions for the isolation and/or treatment of a test sample (e.g., pretreatment reagents), may also be included in the kit. The kit may additionally include one or more other controls. One or more of the components of the kit may be lyophilized and the kit may further comprise reagents suitable for the reconstitution of the lyophilized components.

The various components of the kit optionally are provided in suitable containers. As indicated above, one or more of the containers may be a microtiter plate. The kit further can include containers for holding or storing a sample (e.g., a container or cartridge for a blood or urine sample). Where appropriate, the kit may also optionally contain reaction vessels, mixing vessels and other components that facilitate the preparation of reagents or the test sample. The kit may also include one or more instruments for assisting with obtaining a test sample, such as a syringe, pipette, forceps, measured spoon, or the like.

As used herein, the term “about”, in the context of level of DNA-methylation, typically means +/−5% of the stated value, more typically +/−4% of the stated value, more typically +/−3% of the stated value, more typically, +/−2% of the stated value, even more typically +/−1% of the stated value, and even more typically +/−0.5% of the stated value.

As used herein, the term “one or more” refers to one, two, there, four, five, six, seven, eight, nine, ten or more possible probes or any other feature that is recited as “one or more”.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and sub-generic groupings falling within the generic disclosure also form part of the invention. This includes the generic description of the invention with a proviso or negative limitation removing any subject matter from the genus, regardless of whether or not the excised material is specifically recited herein.

### EXPERIMENTAL SECTION

### Materials and Methods

Clinical samples: Women with euploidy and Down syndrome (DS) (also known as Trisomy 21, or T21) pregnancies, who attended KK Women's and Children's Hospital, Singapore, were recruited. Informed consent was obtained under the ethics approval from the SingHealth CRIB Committee.

Ten mL of peripheral blood from each subject was collected into EDTA tubes. The blood samples were centrifuged at 1,790 g for 10 min at 4° C. After removing the supernatant plasma, the blood cells were transferred to a new microcentrifuge tube and centrifuged at 2,300 g for 5 min at room temperature to remove the residual plasma. The blood cells containing buffy coat were then collected and stored at −0.80° C. DNA was extracted from 200 μL of blood cells from pregnancies using QIAamp DNA Blood Mini Kit (QIAGEN GmbH, Germany), according to manufacturer's instructions. DNA samples eluted with 50 μL of DNase and RNase-free water (Sigma) were stored at −80° C.

Chorionic villus samples from subjects carrying a normal or DS foetus at the first or second trimesters of pregnancy were collected by chorionic villus sampling (CVS). Placenta villi samples (foetal side) from DS foetuses were collected from termination of pregnancy (TOP). All tissue samples were washed with diethylpyrocarbonate (Sigma-Aldrich, USA) treated water. Tissues were stored at −80° C. for DNA analysis. Genomic DNA extraction from tissues was performed with QIAamp DNA Mini Kit (QIAGEN GmbH, Germany), according to manufacturer's instructions.

### Example 1

### Discovery of DNA Methylation Biomarkers

The maternal plasma DNA from peripheral blood of a pregnant woman contains both maternal DNA (derived primarily from leukocytes) and foetal DNA (derived from placental cells). Foetal DNA constitutes about 10% of all cell-free DNA in maternal-plasma. One can distinguish foetal and maternal DNA based on DNA methylation differences of specific genomic regions between foetal and maternal DNA. DNA methylation differences are also present between normal and disease fetuses in placenta DNA. In some genomic regions DNA methylation levels are higher in disease samples while in other regions DNA methylation levels are lower in disease samples compared with normal samples.

Reduced representation bisulfite sequencing (RRBS) was used for quantifying genome-wide DNA methylation profiles in normal and Trisomy 21 placenta samples. Two restriction enzymes (TaqαI and MspI, both from New England Biolabs) were used to digest the genomic DNA samples. DNA fragments were purified with the QIAquick PCR Purification Kit (QIAGEN GmbH), and were end-repaired, 3′-end-adenylated, and adapter-ligated using ChIP-Seq Sample Preparation Kit (Illumina, USA). Illumina's RRBS for Methylation Analysis protocol was followed, except that 10 μL the methylation adapter oligonucleotides were used and the ligation was performed for 15 min at 20° C. in the adapter-ligation step. Two different sizes of fragments (150-197 by and 207-230 bp) were selected by gel electrophoresis with a 3% agarose gel. The purified fragments were then bisulfite treated using the EZ DNA Methylation-Gold Kit (Zymo Research, USA). The converted DNA was amplified using HotStarTaq DNA Polymerase Kit (QIAGEN GmbH), with 1× reaction buffer, 1.5 mM of additional MgCl2, 300 μM of dNTP mix, 500 nM each of PCR primer PE 1.0 and 2.0, and 2.5 U of HotStarTaq DNA polymerase. The thermocycling condition was 15 min at 94° C. for heat activation, and 8-12 cycles of 20 sec at 94° C., 30 sec at 65° C. and 30 sec at 72° C., followed by a 5 min final extension at 72° C. The amplified fragments were purified by gel electrophoresis and further quantified by the Agilent 2100 Bioanalyzer (Agilent Technologies, USA). Each DNA library was analyzed by two lanes of paired-end sequencing (2×36 bp) read on an Illumina Genome Analyzer IIx.

Sequencing data was analyzed. The human genome was converted into two reference genomes for sequencing alignment. The C2T converted reference genome was derived by converting all cytosines to thymines. The G2A converted reference genome was derived by converting all guanines to adenosines. After initial quality control based on their Phred scores (Ewing et al. Genome Res 1998; 8 (3): 186-94) and fragment ending with expected tri-nucleotides after enzymatic reaction, the sequencing reads were aligned to two reference genomes separately using Bowtie aligner (Langmead et al. Genome Biol 2009; 10 (3): R25). The newly added cytosines in the “end-repair” step were excluded from methylation analysis and CpGs overlapping with potential polymorphisms were also excluded. Methylation level of each CpG site was calculated as:

Methylation level for a CpG=Count of Cytosine/(Count of Cytosine+Count of Thymine)*100%.

### Example 2

### T21 Foetus Detection Using Methylation Biomarkers

FIG. 1 shows a schematic describing the following steps. Step 1: Removal of unmethylated DNA for selected biomarkers by methylation-sensitive restriction enzymes. In the case of a foetal/maternal DNA mixture experiment, this step removed maternal DNA background since the biomarkers regions were mostly unmethylated. 25 ng of genomic DNA was subjected to methylation-sensitive restriction enzyme digestion in a 15 μL system, containing 1×buffer 4, 1×BSA, 9 units of BstUI, 10 units of HpaII and 10 units of HhaI (New England Biolabs, USA). Mock digestion without restriction enzymes was set up as control. The samples were incubated at 37° C. for 2 hr and then 60° C. for 2 hr.

Step 2: Exonuclease I treatment was used to remove the 3′ overhang for the digested DNA. 10 units of Exonuclease I (New England Biolabs, USA) was added to the enzyme digested sample, and incubated at 37° C. for 1 hr, followed by heat inactivation at 80° C. for 20 min.

Step 3: Denaturation of the genomic DNA and probe hybridization. A mixture of probe sets containing 1000 amole (atto mole) of each probe set was added to samples from Step 2. Each probe set contains 2 probes. The first probe contained three sequences: a sequence for the qPCR forward primer (in bold), a sequence for the TaqMan probe (underlined) (for Group 1 biomarkers:

(SEQ ID NO: 127 and SEQ ID NO: 123)
5′-GCATGGCTGCTGAGATCGTTCCACAGTATGAATCTCT-3′;

for Group 2 biomarkers:

(SEQ ID NO: 127 and SEQ ID NO: 124))
5′-GCATGGCTGCTGAGATCGTTCCACACATAGAGTTCTT-3′,

and a biomarker-specific sequence. The second probe contained two sequences, a sequence for the qPCR reverse primer (5′-TCGATGCGAACGTGCG-3′(SEQ ID NO: 135)) and a biomarker-specific sequence. The second probe is phosphorylated at the 5′ end and with an optional 3′ Biotin-TEG modification (Integrated DNA technologies, USA). The sample was then incubated at 95° C. for 10 min to denature the genomic DNA, followed by incubation at 60° C. for 16-18 hr for probe hybridization.

Step 4: Ligation of annealed probes. When the two probes from each probe set were hybridized to their target sequences, they were ligated in a 20 μL system, containing 18.5 mM Tris, 41.9 mM potassium acetate, 9.3 mM magnesium acetate, 10 mM DTT, 1 mM NAD; 0.02% Triton X-100, and 20 units of Taq DNA ligase (New England Biolabs, USA), at 60° C. for 2 hr.

Step 5: Beads purification to remove excess of probes. After ligation, the excess of probes were removed either by Agencourt AMPure XP beads (Beckman Coulter, USA) or by Dynabeads MyOne Streptavidin C1 beads (Life Technologies, USA), according to manufacturer's instructions.

Step 6: Detection of methylated foetal DNA by quantitative real-time PCR (qPCR). Beads purified DNA from Step 5 was then subjected to qPCR to detect methylated foetal DNA. Each reaction contains 1×TaqMan Universal PCR Master Mix (Life Technologies, USA), 300 nM each of forward primer (5′-GCATGGCTGCTGAGATCGT-3′; SEQ ID NO: 127) and reverse primer (5′-CGCACGTTCGCATCGA-3′; SEQ ID NO: 128), 100 nM each of TaqMan probes (Group 1 biomarkers: 5′-FAM-CCACAGTATGAATCTCT-MGB-3′(SEQ ID NO: 125); Group 2 biomarkers: 5′-VIC-CCACACATAGAGTTCTT-MGB-3′ (SEQ ID NO: 126)) (Life Technologies, USA), and DNA from Step 5. The qPCR assays were performed in the ABI 7500 Real-Time PCR System (Life Technologies, USA). The thermo profile is 50° C. for 2 min, and 95° C. heat activation for 10 min, followed by 50 cycles of 95° C. for 15 sec and 60° C. for 1 min. Result was analyzed by 7500 Software v2.0.1.

### Example 3

### T21 Foetus Detection Using Methylation Biomarkers

Ten mL of peripheral blood from each subject was collected into EDTA tubes. The blood samples were centrifuged at 1,790 g for 10 min at 4° C. The supernatant was transferred to a new microcentrifuge tube and centrifuged at 16,100 g for 10 min at 4° C. The supernatant cell-free plasma was then collected and stored at −80° C. DNA was extracted from 1.6 mL of plasma from pregnancies using QIAamp DNA Blood Mini Kit (QIAGEN GmbH, Germany), according to manufacturer's instructions. DNA samples eluted with 75 μL of DNase and RNase-free water (Sigma) were stored at −80° C.

Step 1: Removal of unmethylated DNA for selected biomarkers by methylation-sensitive restriction enzymes. In the case of a foetal/maternal DNA mixture experiment, this step removed maternal DNA background since the biomarker regions were mostly unmethylated. Half of genomic DNA extracted from maternal plasma was subjected to methylation-sensitive restriction enzyme digestion in a 45 μL system, containing 1×buffer 4, 1×BSA, 20 units of BstUI, 20 units of HpaII and 20 units of HhaI (New England Biolabs, USA). Mock digestion without restriction enzymes was set up as control. The samples were incubated at 37° C. for 2 hr and then 60° C. for 2 hr.

Step 2: Detection of methylated foetal DNA by quantitative real-time PCR (qPCR). Restriction enzyme digested DNA from Step 1 was then subjected to qPCR to detect methylated foetal DNA. Two biomarkers were assayed, assay 1 (chr15:78,933,445-78,933,521) from Group 1 and assay 2 (chr19:59,025,557-59,025,614) from Group 2. Assay 1 reaction contains 1×TaqMan Universal PCR Master Mix (Life Technologies, USA), 300 nM each of forward primer (5′-ACCCCACAGCGGAGCTC-3′; SEQ ID NO: 131) and reverse primer (5′-AGAAAAGGACCAGGGAAGGC-3′; SEQ ID NO: 133), 200 nM of TaqMan probe (5′-FAM-CGGCTGCCACCCG-MGB-3′; SEQ ID NO: 129) (Life Technologies, USA), and 10 μL of DNA in a 50 μL system. Assay 2 reaction contains 1×TaqMan Universal PCR Master Mix (Life Technologies, USA), 300 nM each of forward primer (5′-AACACATGGTCACGCACACC-3′; SEQ ID NO: 132) and reverse primer (5′-CGCTTGGCGCAGACG-3′; SEQ ID NO: 134), 150 nM of TaqMan probe (5′-VIC-CGCGCCTTCCAGTG-MGB-3′; SEQ ID NO: 130) (Life Technologies, USA), and 10 μL of DNA in a 50 μL system. The qPCR assays were performed in the ABI 7500 Real-Time PCR System (Life Technologies, USA). The thermo profile is 50° C. for 2 min, and 95° C. heat activation for 10 min, followed by 50 cycles of 95° C. for 15 sec and 60° C. for 1 min. Result was analyzed by 7500 Software v2.0.1.

### Results

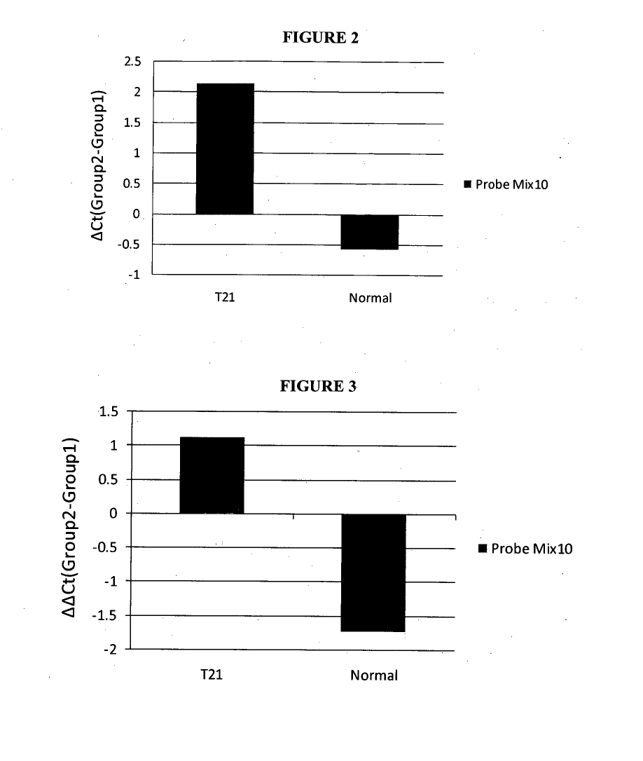

Different combinations of probe mixtures were examined with genomic DNA from one normal and one T21 cases. Cycle Threshold (Ct) values for Group 1 and Group 2 biomarkers were determined in qPCR. The signal ratio for Group 1 and Group 2 was determined by calculating the Ct difference (ΔCt). ΔCt=Ct(Group 2)−Ct(Group 1) where a higher ΔCt value is expected in T21 samples as compared to normal samples. FIG. 2 illustrates the methylation difference between Group 1 and Group 2 biomarkers in normal and T21 samples using probe mix 10, which contains 35 biomarkers from Group 1 and 26 biomarkers from Group 2. The details of probe sequences and their target biomarkers are listed in Mix10 Group 1 (see Table 7) and Mix10 Group 2 (see Table 8).

Alternatively, a mock digestion was performed for each sample. A mock digestion was exactly the same as the real digestion (specified in Steps 1-6 in Example 2), except no restriction enzyme was added in Step 1 and no Exonuclease I was added in Step 2. Ct difference between enzyme-digested sample and its mock-digested control was calculated where for each group (Group 1 and Group 2) ΔCt=Ct (“enzyme-digested”)−Ct(“mock-digested-control”). This ΔCt value represents DNA methylation level for all measured biomarkers in each Group. The difference of the ΔCt (“enzyme-digested”−“mock-digested-control”) values between Group 1 and Group 2 biomarkers were then compared to obtain ΔΔCt (Group2−Group 1). The calculated ΔΔCt (Group2−Group 1) value represents the ratio of targeted methylated DNA in Group 1 and Group 2. FIG. 3 shows the ΔΔCt (Group2−Group1) values from probe mix10, whose methylation difference between normal and T21 tissues is the biggest among all combinations of probe mixtures tested.

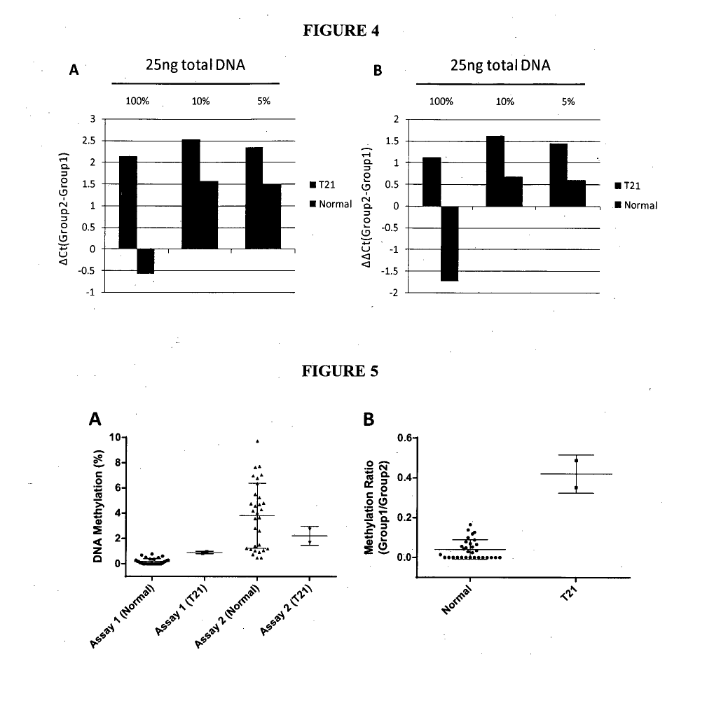

DNA samples obtained from maternal plasma in first trimester contain roughly 10% of foetal DNA and 90% of maternal DNA. To mimic maternal plasma samples, we generated two types of DNA mixture samples, with foetal DNA at 10% and 5% of total DNA, respectively. We used purified placental DNA (foetal origin) or CVS DNA and purified peripheral blood DNA from pregnant women, as these two tissues are the main contributors of foetal and maternal DNA in maternal plasma during pregnancy. As shown in FIG. 4, the same mix 10 probe sets was used. The sample spiked in with T21 placenta DNA (mimicking a maternal plasma sample from a woman pregnant with a T21 foetus) was clearly different from the sample spiked in with normal CVS DNA (mimicking a maternal plasma sample from a woman pregnant with a non-T21 foetus).

Two biomarkers were examined with genomic DNA extracted from maternal plasma samples from pregnancies carrying normal (N=31) or T21 (N=2) foetus. Assay 1 represents biomarkers from Group 1 where T21 cases yield higher signal than normal cases, and assay 2 represents biomarkers from Group 2 where normal cases yield higher signal than T21 cases. Methylation-sensitive restriction enzyme digestion was performed to remove unmethylated foetus signal as well as maternal background. A mock digestion was performed for each sample as control. Ct difference between enzyme-digested sample and its mock-digested control was calculated where for each group (Group 1 and Group 2) ΔCt Ct(“enzyme-digested”)−Ct(“mock-digested-control”). DNA methylation level was than calculated as following: DNA methylation (%)=2^{-[Ct(“enzyme-digested”)-Ct(“mock-digested-control”)]}×100%.

The methylation difference between biomarkers from Group 1 and Group 2 was obtained by calculating the methylation ratio of Group 1 and Group 2. FIG. 5 shows the DNA methylation level in the examined biomarkers and the methylation ratio of Group 1 and Group 2 from maternal plasma samples, demonstrating higher values of methylation ratio of Group 1 and Group 2 in T21 samples than in normal samples.

1.-13. (canceled)

14. A method of determining the likelihood of a foetus to suffer from trisomy 21 or partial trisomy 21, comprising the steps of:
a) providing an isolated total DNA sample from a pregnant woman, comprising foetal DNA and maternal DNA;
b) removing maternal DNA background;
c) measuring a signal indicative for the level of foetal DNA based on one or more biomarkers/biomarker regions listed in any one of Tables 1 to 8, where in the case where the maternal DNA background had a level of methylation below 10%, the signal is the level of methylated foetal DNA and in the case where the maternal DNA background had a level of methylation above 90%, the signal is the level of unmethylated foetal DNA;
d) determining a ratio of signals obtained under step c) by dividing the signals of one or more of Group 1 and/or Group 3 biomarkers/biomarker regions over the signals of one or more of Group 2 and/or Group 4 biomarkers/biomarker regions, wherein a ratio higher than the ratio determined in control foetal DNA obtained from a non-diseased foetus indicates that the foetus is likely to suffer from trisomy 21 or partial trisomy 21;
wherein each of the groups is characterized by:
Group 1: biomarker/biomarker region listed in Table 1 (Group 1), Table 5 (Group 1′), or Table 7 (Mix10 Group 1);
Group 2: biomarker/biomarker region listed in Table 2 (Group 2) or Table 6 (Group 2′), or Table 8 (Mix10 Group 2);
Group 3: biomarker/biomarker region listed in Table 3 (Group 3); and
Group 4: biomarker/biomarker region listed in Table 4 (Group 4).

15. The method according to claim 14, wherein the isolated total DNA from step (a) is obtained from the group consisting of a bodily fluid or a tissue sample obtained from the pregnant woman.

16. The method according to claim 15, wherein the bodily fluid is selected from the group consisting of whole blood, saliva, urine and amniotic fluid.

17. The method according to claim 16, wherein the bodily fluid is whole blood comprising blood cells, plasma and serum.

18. The method according to claim 15, wherein the total DNA is obtained from plasma or serum.

19. The method according to claim 15, wherein the tissue is selected from the group consisting of placental tissue and amniotic sac tissue.

20. The method according to claim 14, wherein the maternal DNA is maternal peripheral blood DNA.

21. The method according to claim 14, wherein step (b) is performed by treating the total isolated DNA with a reagent that differentially modifies methylated or non-methylated DNA.

22. The method according to claim 21, wherein the reagent is selected from the group consisting of sodium bisulfite, one or more enzymes that only cleaves methylated DNA and one or more enzymes that only cleaves non-methylated DNA.

23. The method according to claim 22, wherein the enzyme is selected from the group consisting of MspJI, LpnPI, FspEI, DpnI, DpnII, McrBC, MspI, HapII, AatII, AciI, AclI, AfeI, AgeI, AscI, AscI, AsiSI, AvaI, BceAI, BmgBI, BsaAI, BsaHI, BsiEI, BsiWI, BsmBI, BspDI, BsrFI, BssHII, BstBI, BstUI, Clal, EagI, FauI, FseI, FspI, HaeII, HgaI, HhaI, HinP1I, HpaII, Hpy99I, HpyCH4IV, KsaI, MluI, NaeI, NarI, NgoMIV, NotI, NruI, Nt.BsmAI, NtCviPII, PaeR7I, PmlI, PvuI, RsrII, SacII, SalI, SfoI, SgrAI, SmaI, TspMI and ZraI.

24. The method according to claim 14, wherein prior to step (c), the total DNA is treated with an enzyme which catalyses the removal of nucleotides from single-stranded DNA in the 3′ to 5′ direction.

25. The method according to claim 14, wherein the total DNA is incubated with one or more probe sets.

26. The method according to claim 25, wherein each probe set comprises:
(a) a first probe, comprising a sequence for binding a forward primer, a sequence for binding a third probe and a sequence for binding to the one or more biomarker/biomarker regions; and
(b) a second probe, comprising a sequence for binding a reverse primer and a sequence for binding to the one or more biomarker/biomarker regions.

27. The method according to claim 26 wherein the second probe is phosphorylated at the 5′ end.

28. The method according to claim 26, wherein the binding sequence for the third probe is different for each of biomarker/biomarker region groups 1 to 4.

29. The method according to claim 28, wherein the binding sequence for the third probe for the Group 1 biomarker/biomarker region comprises the sequence
(SEQ ID NO: 123)
5′-CCACAGTATGAATCTCT-3′.

30. The method according to claim 28, wherein the binding sequence for the third probe for the Group 2 biomarker/biomarker region comprises the sequence
(SEQ ID NO: 124)
5′-CCACACATAGAGTTCTT-3′.

31. The method according to claim 26, wherein the sequences of the first probe and second probe in each probe set is selected from any one of the probe sets listed in Tables 7 or 8.

32. The method according to claim 26, wherein the two probes from each probe set are ligated together.

33. The method according to claim 32, further comprising the step of removing the excess probes which have not been ligated together.

34. The method according to claim 32, wherein the step of removing the excess probes is performed using bead purification.

35. The method according to claim 14, wherein the signal which is indicative of the level of foetal DNA in step (c) is a fluorescent signal.

36. The method according to claim 35, wherein a different fluorescent signal is measured for each of biomarker/biomarker region groups 1 to 4.

37. The method according to claim 35, wherein the fluorescent signals originate from one or more probes having fluorophores thereon.

38. The method according to claim 26, wherein the forward primer comprises the sequence selected from the group consisting of 5′-GCATGGCTGCTGAGATCGT-3′ (SEQ ID NO: 127).

39. The method according to claim 26, wherein the reverse primer comprises the sequence selected from the group of 5′-CGCACGTTCGCATCGA-3′ (SEQ ID NO: 128).

40. The method according to claim 26, wherein the third probe comprises the sequence selected from the group consisting of 5′-FAM-CCACAGTATGAATCTCT-MGB-3′ (SEQ ID NO: 125).

41. The method according to claim 26, wherein the third probe comprises the sequence selected from the group consisting of 5′-VIC-CCACACATAGAGTTCTT-MGB-3′ (SEQ ID NO: 126).

42. The method according to claim 26, wherein the signal indicative of the level of foetal DNA in step (c) is measured by quantitative polymerase chain reaction.

43.-46. (canceled)

47. A method of determining the methylation levels of a biomarker/biomarker region comprising the steps of:
(a) treating a sample comprising both foetal and maternal DNA with a reagent that differentially modifies methylated and non-methylated DNA;
(b) calculating the percentage of unmodified cytosine residues over the total number of modified and unmodified cytosine residues in order to determine the methylation levels of a biomarker/biomarker region.

48. The method according to claim 47, wherein the reagent in step (a) is selected from the group consisting of sodium bisulfite, one or more enzymes that preferentially cleaves methylated DNA and one or more enzymes that preferentially cleaves non-methylated DNA.

49. The method according to claim 47, further comprising bisulfite sequencing prior to step (b).

50. The method according to claim 49, where signals detected from the unmodified cytosine residues and the modified cytosine residues are compared to calculate the methylation level.
